SEQUENCE LISTING (31 sequences)

SEQ ID NO: 1 - protein, 1368 aa, synthetic construct [sequence omitted]

SEQ ID NO: 2 - protein, 325 aa, synthetic construct
LPAWHKSIVSFRQLREEVLGELSNVEQAAGLRWSRFPDLNRILKGHRKGELTVFTGPTGSGKTTFISEYALDLCSQGVNTLWGSFEISNVRLARVMLTQFAEGRLEDQLDKYDHWADRFEDLPLYFMTFHGQQSIRTVIDTMQHAVYVYDICHVIIDNLQFMMGHEQLSTDRIAAQDYIIGVFRKFATDNNCHVTLVIHPRKEDDDKELQTASIFGSAKASQEADNVLILQDRKLVTGPGKRYLQVSKNRFDGDVGVFPLEFNKNSLTFSIPPKNKARLKKIKDDTGPVAKKPSSGKKGATTQNSEICSGQAPTPDQPDTSKRSK

SEQ ID NO: 3 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 236 aa, synthetic construct
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 6 - protein, 837 aa, synthetic construct
MGIQGLLQFIQEASEPVNVKKYKGQAVAVDTYCWLHKGAIACAEKLAKGEPTDRYVGFCMKFVNMLLSYGVKPILIFDGCTLPSKKEVERSRRERRQSNLLKGKQLLREGKVSEARDCFARSINITHAMAHKVIKAARALGVDCLVAPYEADAQLAYLNKAGIVQAVITEDSDLLAFGCKKVILKMDQFGNGLEVDQARLGMCKQLGDVFTEEKFRYMCILSGCDYLASLRGIGLAKACKVLRLANNPDIVKVIKKIGHYLRMNITVPEDYITGFIRANNTFLYQLVFDPIQRKLVPLNAYGDDVNPETLTYAGQYVGDSVALQIALGNRDVNTFEQIDDYSPDTMPAHSRSHSWNEKAGQKPPGTNSIWHKNYCPRLEVNSVSHAPQLKEKPSTLGLKQVISTKGLNLPRKSCVLKRPRNEALAEDDLLSQYSSVSKKIKENGCGDGTSPNSSKMSKSCPDSGTAHKTDAHTPSKMRNKFATFLQRRNEESGAVVVPGTRSRFFCSSQDFDNFIPKKESGQPLNETVATGKATTSLLGALDCPDTEGHKPVDANGTHNLSSQIPGNAAVSPEDEAQSSETSKLLGAMSPPSLGTLRSCFSWSGTLREFSRTPSPSASTTLQQFRRKSDPPACLPEASAVVTDRCDSKSEMLGETSQPLHELGCSSRSQESMDSSCGLNTSSLSQPSSRDSGSEESDCNNKSLDNQGEQNSKQHLPHFSKKDGLRRNKVPGLCRSSSMDSFSTTKIKPLVPARVSGLSKKSGSMQTRKHHDVENKPGLQTKISELWKNFGFKKDSEKLPSCKKPLSPVKDNIQLTPETEDEIFNKPECVRAQRAIFH

SEQ ID NO: 7 - protein, 291 aa, synthetic construct
MSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 8 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 9 - protein, 19 aa, synthetic construct
MKRTADGSEFESPKKKRKV

SEQ ID NO: 10 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 11 - DNA, 19 bp, synthetic construct
  source 1..19: mol_type = other DNA
acatgaggatcacccatgt

SEQ ID NO: 12 - DNA, 7568 bp, synthetic construct
  source 1..7568: mol_type = other DNA
gaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatattgagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcatgaaaagaaccgctgatggcagcgagttcgagagccctaagaagaagcggaaggtgatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcggcagcaaaagaaccgccgacggctcagagttcgagcccaaaaagaagcggaaagtgtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 13 - DNA, 5390 bp, synthetic construct
  source 1..5390: mol_type = other DNA
gaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatattgagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcatgaaaagaaccgctgatggcagcgagttcgagagccctaagaagaagcggaaggtggagactctccaagccttggatatgccagtgttgcctgtaactgcaactgaaatccgccagtatttgcgggggcatgggatccccttccaggatggtcacagttgcctgcgggcactgagcccctttgcagagtcttcacagctcaaaggccagactggtgttaccacttccttcagcctcttcattgacaagaccacaggccactttctctgcatgaccagcctagcagaagggagctgggaagacttccaggccagcgtggaggggcgaggggatggggccagggaggggtttctgcttagcaaggcaccagaatttgaggacagcgaggaggtccggaggatctggaaccgagcaatacctctctgggagctgcctgatcaggaggaggttcagctggctgatacaatgtttggccttaccaaggttacagatgacacactcaagcgtttcagtgtgcgatatctgcgacctgctcgcagtcttgtcttcccttggttctcccctgggggctcaggattacgaggcctgaagctcctagaggctaaatgccagggggatggagtgagctacgaggaaaccactattccccgacccagcgcctaccacaatctgtttggattaccactgattagtcgtcgagatgctgaggtggtactgacgagtcgtgagcttgacagcctggccttgaaccagtccacggggctgcctacccttactctaccccgaggaacgacctgcttaccccctgccttactcccttacctggaacagttccggcggattgtattctggttgggggatgaccttcggtcctgggaagccgccaagttgtttgcacgaaaactgaaccccaaacgatgcttcttggtgcgaccaggagaccagcaaccccgtcccctggaggccctgaacggaggcttcaatctttctcgtattcttcgtaccgccctgcctgcctggcacaagtccatcgtatctttccggcagcttcgggaggaggtgctaggagaactgtcaaatgtggagcaagcagctggcctccgctggagccgctttccagacctcaatcgtatcttgaagggacatcgaaagggcgagctgacggtcttcacagggccaacaggcagtggaaagacgacattcatcagtgagtatgccctggatttgtgttcccagggggtgaacacactgtggggtagcttcgagatcagcaatgtgagactagcccgggtcatgctgacacagtttgccgaggggcggctggaagatcaactggacaaatatgatcactgggctgaccgctttgaggacctgcccctctatttcatgactttccatggacagcaaagcatcaggactgtaatagatacaatgcaacatgcagtctacgtctatgacatttgtcatgtgatcatcgacaacctgcagttcatgatgggtcacgagcagctgtccacagacaggatcgcagctcaagactacatcatcggggtctttcggaagtttgcaacagacaataactgccatgtgacactggtcattcacccccggaaagaggatgatgacaaggaactgcagacagcgtccatttttggctcagccaaagcaagccaggaagcagacaatgttctgatcctgcaggacaggaagctggtaaccgggccagggaaacggtatctgcaggtgtccaagaaccgctttgatggagatgtaggtgtcttcccgcttgagttcaacaagaactccctcaccttctccattccaccaaagaacaaggcccggctcaagaagatcaaggatgacactggaccagtggccaaaaagccctcttctggcaaaaagggggctacgacacagaactctgagatttgctcaggccaggcccccactcccgaccagccagacacctccaagcgttcaaagagcggcggcagcaaaagaaccgccgacggctcagagttcgagcccaaaaagaagcggaaagtgtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 14 - DNA, 4172 bp, synthetic construct
  source 1..4172: mol_type = other DNA
gaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatattgagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcatgaaaagaaccgctgatggcagcgagttcgagagccctaagaagaagcggaaggtgatgtccgaaccacctagagccgagaccttcgtgttcctggacctggaggccacaggcctgcccaacatggaccccgagatcgccgaaatcagcctgtttgccgtgcacagaagcagcctggaaaaccctgaaagagatgacagcggcagcctggtgctgcccagagtgctggacaagctgacactgtgtatgtgccccgaacggcctttcaccgccaaggcctctgaaatcaccggactgtcttctgagagcctgatgcactgcggcaaggccggctttaacggcgctgtggtgcggaccctgcagggctttctgtccagacaggagggccccatctgcctggtcgcccacaacggcttcgattacgacttccccctgctctgcaccgagctgcaaagactgggcgcccacctgccacaggacaccgtgtgtctggatacactgcccgccctgcggggcctggacagagcccatagccacggcaccagggcccagggcagaaagagctacagccttgcttctctgttccaccggtacttccaggccgagccctctgccgcccacagcgccgagggagatgtgcacacactgctgctgatcttcctgcacagagctcctgaactgctggcctgggctgatgagcaggcccgcagctgggctcacatcgagcctatgtacgtgcctcctgacggcccttccctggaagccagcggcggcagcaaaagaaccgccgacggctcagagttcgagcccaaaaagaagcggaaagtgtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 15 - DNA, 4604 bp, synthetic construct
  source 1..4604: mol_type = other DNA
gaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatattgagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcatgaaaagaaccgctgatggcagcgagttcgagagccctaagaagaagcggaaggtgatgggaattcaaggcctggccaaactaattgctgatgtggcccccagtgccatccgggagaatgacatcaagagctactttggccgtaaggtggccattgatgcctctatgagcatttatcagttcctgattgctgttcgccagggtggggatgtgctgcagaatgaggagggtgagaccaccagccacctgatgggcatgttctaccgcaccattcgcatgatggagaacggcatcaagcccgtgtatgtctttgatggcaagccgccacagctcaagtcaggcgagctggccaaacgcagtgagcggcgggctgaggcagagaagcagctgcagcaggctcaggctgctggggccgagcaggaggtggaaaaattcactaagcggctggtgaaggtcactaagcagcacaatgatgagtgcaaacatctgctgagcctcatgggcatcccttatcttgatgcacccagtgaggcagaggccagctgtgctgccctggtgaaggctggcaaagtctatgctgcggctaccgaggacatggactgcctcaccttcggcagccctgtgctaatgcgacacctgactgccagtgaagccaaaaagctgccaatccaggaattccacctgagccggattctgcaggagctgggcctgaaccaggaacagtttgtggatctgtgcatcctgctaggcagtgactactgtgagagtatccggggtattgggcccaagcgggctgtggacctcatccagaagcacaagagcatcgaggagatcgtgcggcgacttgaccccaacaagtaccctgtgccagaaaattggctccacaaggaggctcaccagctcttcttggaacctgaggtgctggacccagagtctgtggagctgaagtggagcgagccaaatgaagaagagctgatcaagttcatgtgtggtgaaaagcagttctctgaggagcgaatccgcagtggggtcaagaggctgagtaagagccgccaaggcagcacccagggccgcctggatgatttcttcaaggtgaccggctcactctcttcagctaagcgcaaggagccagaacccaagggatccactaagaagaaggcaaagactggggcagcagggaagtttaaaaggggaaaaagcggcggcagcaaaagaaccgccgacggctcagagttcgagcccaaaaagaagcggaaagtgtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 16 - protein, 5 aa, synthetic construct
KKRKV

SEQ ID NO: 17 - protein, 7 aa, synthetic construct
PKKKRKV

SEQ ID NO: 18 - protein, 16 aa, synthetic construct
KRPAATKKAGQAKKKK

SEQ ID NO: 19 - protein, 18 aa, synthetic construct
KRTADGSEFESPKKKRKV

SEQ ID NO: 20 - protein, 29 aa, synthetic construct
DSLLMNRRKFLYQFKNVRWAKGRRETYLC

SEQ ID NO: 21 - protein, 4 aa, synthetic construct
GGGS

SEQ ID NO: 22 - protein, 5 aa, synthetic construct
GGGGS

SEQ ID NO: 23 - protein, 5 aa, synthetic construct
EAAAK

SEQ ID NO: 24 - protein, 4 aa, synthetic construct
SGGS

SEQ ID NO: 25 - protein, 16 aa, synthetic construct
SGSETPGTSESATPES

SEQ ID NO: 26 - DNA, 23 bp, synthetic construct
  source 1..23: mol_type = other DNA
ggcccagactgagcacgtgatgg

SEQ ID NO: 27 - DNA, 33 bp, synthetic construct
  source 1..33: mol_type = other DNA
gagcacgagatggcaaaggaaaggaagccctgc

SEQ ID NO: 28 - DNA, 63 bp, synthetic construct
  source 1..63: mol_type = other DNA
gtcatcttagtcattaccccttgaggtgttcgttgtaactcatataaactgagttcccatgtt

SEQ ID NO: 29 - DNA, 23 bp, synthetic construct
  source 1..23: mol_type = other DNA
gcattttcaggaggaagcgatgg

SEQ ID NO: 30 - DNA, 287 bp, synthetic construct
  source 1..287: mol_type = other DNA
ctagaggggtgaggctgaaacagtgacctgtcttggttttcgctccgaaggtaaaagaaatcattgagtcccccgccttcagaagagggtgcattttcaggaggaaggatggcttcagacagcatatttgagtcatttccttcgtacccacagtgcttcatgagaggtgagtacatgctggtcttgtaatatctacttttgctcagctttgcctgtaatgaaatggcagcttgtttcacctcggtgcagagatgcctcggtgcctgccagttccctgtcttgtttgt

SEQ ID NO: 31 - DNA, 287 bp, synthetic construct
  source 1..287: mol_type = other DNA
acaaacaagacagggaactggcaggcaccgaggcatctctgcaccgaggtgaaacaagctgccatttcattacaggcaaagctgagcaaaagtagatattacaagaccagcatgtactcacctctcatgaagcactgtgggtacgaaggaaatgactcaaatatgctgtctgaagccatccttcctcctgaaaatgcaccctcttctgaaggcgggggactcaatgatttcttttaccttcggagcgaaaaccaagacaggtcactgtttcagcctcacccctctag

(19) United States
(12) Patent Application Publication
(10) Pub. No.: US 2026/0275310 A1
(43) Pub. Date: Sep. 17, 2026

(54) Strand Displacement-Based Gene Editing Method and System and Use Thereof

(71) Applicant: Beijing Qi Biodesign Biotechnology Company Limited, Beijing (CN)

(72) Inventors: Kevin T. ZHAO, Beijing (CN); Jiacheng HU, Beijing (CN)

(21) Appl. No.: 19/462,424

(22) Filed: Jan. 28, 2026

(30) Foreign Application Priority Data
Dec. 1, 2024 (CN) 202411753597.7
Dec. 31, 2025 (CN) 202512059837.4

(51) Int. Cl. C12N 9/22; C12N 5/071; C12N 15/10; C12N 15/113; C12N 15/52

(52) U.S. Cl. CPC C12N 9/222; C12N 5/0602; C12N 15/102; C12N 15/113; C12N 15/52; C12Y 301/11001; C12Y 301/11002; C12Y 301/11003; C12Y 306/04012; C12N 2310/20; C12N 2510/00

(57) ABSTRACT

The present disclosure belongs to the field of genetic engineering. The present disclosure relates to a strand displacement-based gene editing method and system, and use thereof. Specifically, the present disclosure relates to a gene editing method based on strand displacement mediated by the Twinkle protein, and a gene editing system based on the strand displacement, and use thereof.

### INCORPORATION BY REFERENCE OF SEQUENCE LISTING PROVIDED ELECTRONICALLY

This application contains a Sequence Listing that has been submitted electronically and is hereby incorporated by reference in its entirety. The Sequence Listing was created on Jan. 27, 2026, is named “26-0087-US_SequenceListing.xml,” and is 56,396 bytes in size.

### CROSS-REFERENCE TO RELATED APPLICATIONS

The present disclosure claims the benefits of priority of the Chinese Patent Application No. 202411753597.7, filed on Dec. 1, 2024 and entitled “STRAND DISPLACEMENT-BASED GENE EDITING METHOD AND SYSTEM AND USE THEREOF” and the Chinese Patent Application No. 202512059837.4, filed on Dec. 31, 2025 and entitled “STRAND DISPLACEMENT-BASED GENE EDITING METHOD AND SYSTEM AND USE THEREOF”, the entire contents of which are incorporated herein by reference.

### TECHNICAL FIELD

The present disclosure belongs to the field of genetic engineering. Specifically, the present disclosure relates to a gene editing method based on strand displacement mediated by the Twinkle protein, and a gene editing system based on the strand displacement, and use thereof.

### BACKGROUND

Precise editing of target nucleotide regions has currently been a major research topic in the field of genetic engineering, and offers broad application prospects for advancing the genetics and its applications, particularly for biomedical research, plant genetic improvement, etc.

At present, an ideal genome editing technology should be characterized by: 1) a high gene-editing efficiency; 2) a minimal off-target activity; 3) a broad editing capacity for almost all sites in the genome; and 4) the ability to accomplish different types of gene editing (base substitutions, DNA insertions, DNA deletions, DNA replacements, etc.).

Early genome engineering tools, such as zinc finger nuclease (ZFN), transcription activator-like effector nuclease (TALEN), and CRISPR effector protein (CRISPR/Cas), achieve gene editing by cleaving the nucleotide sequence in a sequence-specific manner, which is then repaired by causing a DNA mutation at the cleavage site via Non-homologous End Joining (NHEJ) or replacing DNA around the cleavage site via Homology Directed Repair (HDR).

With the iteration of technology, Base Editors have emerged that enable precise editing of the target base by virtue of the deamination of nucleoside deaminase. According to the types of base on which the Base Editors acts, the Base Editors can be classified into a cytosine base editor (CBE) and an adenine base editor (ABE). Of these, the CBE can convert the C-G base pair on the double-stranded DNA into the T-A base pair; and the ABE can convert the A-T base pair on the double-stranded DNA into the G-C base pair.

However, the applications of the Base Editor are constrained as a result of limited types of base substitution, the “bystander effect” on non-target bases, the fact that the editable sites can only be within 15±2 nucleotides upstream of the PAM sequence, and the like. In 2019, a Prime Editor was developed. This system includes a fusion protein containing a reverse transcriptase and a Cas protein, and achieves all of 12 possible types of base substitution in the cell genome by reverse transcribing the template sequence via the reverse transcriptase. In spite of this, the Prime Editor still has problems in editing efficiency and other aspects, which restrict their application potential in treatment of diseases, construction of animal models, plant genetics and breeding, etc.

### SUMMARY

The present disclosure provides a gene editing system based on the strand displacement of mitochondrial DNA helicase Twinkle (Twinkle Editor). In human cells, the Twinkle enzyme is a helicase of SF4 superfamily and has a fundamental biological function of acting as a helicase component in the mitochondrial DNA replication complex, where it participates in replication and maintenance of mitochondrial DNA, and frequently functions in the form of a multimer (e.g. hexamer). The biological process where Twinkle is involved in cells further includes: translocations on DNA; DNA replication; DNA strand exchange; and single-strand annealing of DNA. Under the action of the Twinkle enzyme, the present disclosure introduces an exogenous invader DNA strand into the target nucleotide region through a series of strand displacement reactions, thereby achieving precise DNA editing.

The present disclosure provides the following specific technical solutions:

[1]. A method for performing strand displacement-based gene editing in a DNA sequence, the method comprising:
bringing a double-stranded DNA sequence into contact with a sequence-specific nucleic acid nickase to create a nick on the double-stranded DNA sequence;
digesting from the nick with an exonuclease to generate a free single-stranded DNA;
bringing the free single-stranded DNA into contact with an invader DNA and a human mitochondrial DNA helicase Twinkle enzyme; wherein the invader DNA is a single-stranded or double-stranded DNA, and at least one strand of the invader DNA comprises a glue point complementary to the free single-stranded DNA and a displacement template containing a desired nucleotide alteration;
thereby subjecting the glue point of the invader DNA to complementary pairing with the free single-stranded DNA;
thereby subjecting the displacement template of the invader DNA to a strand displacement reaction with an endogenous DNA strand adjacent to a nick site; and
performing DNA repair/replication to introduce the desired nucleotide alteration into the double-stranded DNA sequence.

[2]. The method according to [1], wherein a process of the DNA repair comprises: (1) performing the DNA repair using the displacement template as a template to form a product with the desired nucleotide alteration on two DNA strands; and (2) repairing the nick on DNA.

[3]. The method according to [1], wherein the process of the DNA repair further comprises excising the endogenous DNA strand adjacent to the nick site using a structure-specific nuclease after the strand displacement reaction; wherein the structure-specific nuclease is preferably FEN1.

[4]. The method according to [1], wherein the sequence-specific nucleic acid nickase is a CRISPR-associated protein (Cas) polypeptide having a nickase activity, a zinc finger nuclease (ZFN), a transcription activator-like effector nuclease (TALEN), a meganuclease, a sequence-specific endonuclease, or a variant, fragment and combination thereof.

[5]. The method according to [4], wherein the Cas polypeptide is selected from Cas3, Cas4, Cas5, Cas5e, Cas6, Cas6e, Cas6f, Cas7, Cas8a1, Cas8a2, Cas8b, Cas8c, Cas9, Cas10, Cas10d, Cas12, Cas13, Cas14, CasX, CasF, CasG, CasH, Csy1, Csy2, Csy3, Cse1, Cse2, Cse3, Cse4, Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, TranC, and a variant, fragment or any combination thereof.

[6]. The method according to [1], wherein the sequence-specific nucleic acid nickase comprises a mutant comprising a D10A mutation or an H840A mutation corresponding to the amino acid sequence set forth in SEQ ID NO:1.

[7]. The method according to [1], wherein the Twinkle enzyme is a naturally occurring or truncated Twinkle enzyme; and the Twinkle enzyme comprises a carboxyl-terminal domain (CTD) having an amino acid sequence set forth in SEQ ID NO: 2.

[8]. The method according to [7], wherein a mitochondrial localization signal is removed from the Twinkle enzyme, and then a nuclear localization signal (NLS) is fused to an amino terminus and/or carboxyl terminus of the Twinkle enzyme.

[9]. The method according to [1], wherein the exonuclease is a 3′ exonuclease or a 5′ exonuclease or a combination thereof, wherein the 3′ exonuclease is preferably Trex2; and the 5′ exonuclease is preferably mExol or T5 exonuclease.

[10]. The method according to [1], wherein the desired nucleotide alteration comprises a substitution, insertion or deletion of one or more nucleotides.

[11]. A strand displacement-based gene editing system, comprising components of the strand displacement-based gene editing system and/or an expression construct comprising a nucleotide sequence that encodes the components of the strand displacement-based gene editing system, the components of the strand displacement-based gene editing system comprising:
i) a sequence-specific nucleic acid nickase or a domain thereof that is guided by a guide RNA (sgRNA) sequence to a target DNA;
ii) an exonuclease or a domain thereof;
iii) a human mitochondrial DNA helicase Twinkle enzyme or a domain thereof; and
iv) a single-stranded or double-stranded invader DNA, wherein at least one strand of the invader DNA comprises a glue point complementary to a free single-stranded DNA generated under action of a nickase and the exonuclease, and a displacement template containing a desired nucleotide alteration;
the components being independent of each other, or at least two of the components are linked via a linker to form a fusion protein.

[12]. The strand displacement-based gene editing system according to [11], wherein the components of the strand displacement-based gene editing system further comprise:
v) a structure-specific nuclease;
wherein the structure-specific nuclease is preferably FEN1.

[13]. The strand displacement-based gene editing system according to [11], wherein the sequence-specific nucleic acid nickase is a CRISPR-associated protein (Cas) polypeptide having a nickase activity, a zinc finger nuclease (ZFN), a transcription activator-like effector nuclease (TALEN), a meganuclease, a sequence-specific endonuclease, or a variant, fragment and combination thereof.

[14]. The strand displacement-based gene editing system according to [11], wherein the sequence-specific nucleic acid nickase is the CRISPR-associated protein (Cas) polypeptide having a nickase activity; and the components of the strand displacement-based gene editing system further comprise: vi) a guide RNA that guides a corresponding Cas polypeptide to a target nucleotide region.

[15]. The strand displacement-based gene editing system according to [14], wherein the Cas polypeptide is selected from Cas3, Cas4, Cas5, Cas5e, Cas6, Cas6e, Cas6f, Cas7, Cas8a1, Cas8a2, Cas8b, Cas8c, Cas9, Cas10, Cas10d, Cas12, Cas13, Cas14, CasX, CasF, CasG, CasH, Csy1, Csy2, Csy3, Cse1, Cse2, Cse3, Cse4, Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, TranC, and a variant, fragment or any combination thereof.

[16]. The strand displacement-based gene editing system according to [11], wherein the sequence-specific nucleic acid nickase comprises a mutant comprising a D10A mutation or an H840A mutation corresponding to the amino acid sequence set forth in SEQ ID NO:1.

[17]. The strand displacement-based gene editing system according to [11], wherein the Twinkle enzyme is a naturally occurring or truncated Twinkle enzyme; and the Twinkle enzyme comprises a carboxyl-terminal domain (CTD) having an amino acid sequence set forth in SEQ ID NO: 2.

[18]. The strand displacement-based gene editing system according to [11], wherein the exonuclease is a 3′ exonuclease or a 5′ exonuclease or a combination thereof; wherein the 3′ exonuclease is preferably Trex2; and the 5′ exonuclease is preferably mExol or T5 exonuclease.

[19]. The strand displacement-based gene editing system according to any one of to [18], wherein a nuclear localization signal (NLS) is fused to an amino or carboxyl terminus of at least one of the components.

[20]. The strand displacement-based gene editing system according to any one of to [18], wherein the linker comprises an amino acid sequence (GGGS)n, (GGGGS)n, (G)n, (EAAAK)n, (GGS)n, (SGGS)n or SGSETPGTSESATPES, or a (XP)n motif or a combination thereof, wherein n is independently an integer of 1 to 30, and wherein X is any amino acid.

[21]. The strand displacement-based gene editing system according to any one [11] of to [18], wherein each of the components of the strand displacement-based gene editing system is recruited via a recruitment system that is a MCP-MS2 system or a GCN4-ScFv system.

[22]. A host cell, wherein the host cell comprises the strand displacement-based gene editing system according to any one of [11] to [21].

[23]. A method for generating at least one cell that is genetically modified, wherein the method comprises editing the at least one cell by the method according to any one of [1] to [10], or introducing the strand displacement-based gene editing system according to any one of [11] to [21] into the at least one cell to cause a substitution, insertion or deletion of one or more nucleotides within a target nucleotide editing region in the at least one cell.

[24]. The method according to [23], wherein the method further comprises a step of screening the at least one cell for a cell having one or more desired nucleotide substitutions.

[25]. The method according to [23] or [24], wherein the cell is derived from a prokaryote such as bacteria; or an eukaryote such as plants, fungi or vertebrates.

[26]. The method according to [25], wherein the vertebrates are mammals such as human, mouse, rat, monkey, dog, pig, sheep, cattle, and cat.

[27]. The method according to [25], wherein the plants are crop plants, such as wheat, rice, corn, soybean, sunflower, sorghum, oilseed rape, alfalfa, cotton, barley, millet, sugar cane, tomato, tobacco, cassava or potato.

[28]. Use of the method for performing strand displacement-based gene editing in the DNA sequence according to any one of [1] to [10] or the strand displacement-based gene editing system according to any one of [11] to [21], wherein the use comprises:
a) gene or genome editing;
b) target nucleotide detection and/or diagnosis;
c) editing a target nucleotide sequence to modify an organism or a non-human organism; and
d) treatment of a disease.

[29]. A kit, wherein the kit comprises the strand displacement-based gene editing system according to any one of [11] to [21] or the host cell according to [22].

### Advantageous Effects of the Invention

Compared with the Prime Editor, the Twinkle Editor can directly introduce an exogenous DNA sequence via displacement without requiring a reverse transcription process, thereby improving the gene editing efficiency and reducing imprecise editing caused by errors in the reverse transcription process. Compared with Base Editor, the Twinkle Editor can accomplish more editing types including all of 12 types of base substitutions, DNA insertions, DNA deletions, etc.; furthermore, the invader DNA strand in the Twinkle Editor further contains a glue point complementary to the sequence upstream of the editing region, and only when the glue point undergoes complementary pairing with the target nucleotide can the strand displacement reaction occur, that is, the Twinkle Editor contains two sets of recognition sequences that recognize the target nucleotide region, and thus exhibits a lower off-target activity as compared to the existing gene editing systems.

### BRIEF DESCRIPTION OF THE DRAWINGS

FIG. 1: Schematic diagram for a strand displacement-based gene editing system.

FIG. 2: Schematic diagram for the structure of components of the Twinkle Editor.

FIG. 3: Editing efficiency of the Twinkle Editor in base displacement.

FIG. 4: Editing efficiency of the Twinkle Editor recruited by MCP-MS2 in precise DNA insertion.

FIG. 5: Editing efficiency of the Twinkle Editor with a double-stranded invader DNA template in DNA deletion.

### DETAILED DESCRIPTION

With the goal of enriching the toolkit of the gene editing system, developed are a gene editing method based on strand displacement mediated by the Twinkle protein, and a gene editing system based on the strand displacement, and use thereof. This editing system enables efficient and precise editing of the target nucleotide sequence.

In one aspect of the present disclosure, there is provided a method for performing strand displacement-based gene editing in a DNA sequence, the method comprising:
bringing a double-stranded DNA sequence into contact with a sequence-specific nucleic acid nickase to create a nick on the double-stranded DNA sequence;
digesting from the nick with an exonuclease to generate a free single-stranded DNA;
bringing the free single-stranded DNA into contact with an invader DNA and a human mitochondrial DNA helicase Twinkle; wherein the invader DNA is a single-stranded DNA or a double-stranded DNA, and at least one strand of the invader DNA comprises a glue point complementary to the free single-stranded DNA and a displacement template containing a desired nucleotide alteration;
thereby subjecting the glue point of the invader DNA to complementary pairing with the free single-stranded DNA;
thereby subjecting the displacement template of the invader DNA to a strand displacement reaction with an endogenous DNA strand adjacent to a nick site; and
performing DNA repair/replication to introduce the desired nucleotide alteration into the double-stranded DNA sequence.

In some embodiments, the DNA repair process of the present disclosure comprises: (1) performing the DNA repair using the displacement template as a template to form a product with the desired nucleotide alteration on two DNA strands; and (2) repairing the nick on DNA.

In some embodiments, the DNA repair process of the present disclosure further comprises excising the endogenous DNA strand adjacent to the nick site using a structure-specific nuclease after the strand displacement reaction; wherein the structure-specific nuclease is preferably FEN1.

In some embodiments, the sequence-specific nucleic acid nickase of the present disclosure is a CRISPR-associated protein (Cas) polypeptide having a nickase activity, a zinc finger nuclease (ZFN), a transcription activator-like effector nuclease (TALEN), a meganuclease, a sequence-specific endonuclease, or a variant, fragment and combination thereof.

In some embodiments, the Cas polypeptide of the present disclosure is selected from Cas3, Cas4, Cas5, Cas5e, Cas6, Cas6e, Cas6f, Cas7, Cas8a1, Cas8a2, Cas8b, Cas8c, Cas9, Cas10, Cas10d, Cas12, Cas13, Cas14, CasX, CasF, CasG, CasH, Csy1, Csy2, Csy3, Cse1, Cse2, Cse3, Cse4, Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, TranC, and a variant, fragment or any combination thereof.

In some embodiments, the sequence-specific nucleic acid nickase of the present disclosure comprises a mutant comprising a D10A mutation or an H840A mutation corresponding to the amino acid sequence set forth in SEQ ID NO:1.

In some embodiments, the Twinkle enzyme of the present disclosure is a naturally occurring or truncated Twinkle enzyme; and the Twinkle enzyme comprises a carboxyl-terminal domain (CTD) having an amino acid sequence set forth in SEQ ID NO: 2.

In some embodiments, a mitochondrial localization signal is removed from the Twinkle enzyme of the present disclosure, and then a nuclear localization signal (NLS) is fused to an amino terminus and/or carboxyl terminus of the Twinkle enzyme.

In some embodiments, the exonuclease of the present disclosure is a 3′ exonuclease or a 5′ exonuclease or a combination thereof; wherein the 3′ exonuclease is preferably Trex2; and the 5′ exonuclease is preferably mExol or T5 exonuclease.

In some embodiments, the desired nucleotide alteration of the present disclosure comprises a substitution, insertion or deletion of one or more nucleotides.

In another aspect of the present disclosure, there is provided a strand displacement-based gene editing system, comprising components of the strand displacement-based gene editing system and/or an expression construct comprising a nucleotide sequence that encodes the components of the strand displacement-based gene editing system, the components of the strand displacement-based gene editing system comprising:
i) a sequence-specific nucleic acid nickase or a domain thereof;
ii) an exonuclease or a domain thereof;
iii) a human mitochondrial DNA helicase Twinkle or a domain thereof; and
iv) a single-stranded or double-stranded invader DNA, wherein at least one strand of the invader DNA comprises a glue point complementary to a free single-stranded DNA generated under action of a nickase and the exonuclease, and a displacement template containing a desired nucleotide alteration;
the components being independent of each other, or at least two of the components are linked via a linker to form a fusion protein.

In some embodiments, the components of the strand displacement-based gene editing system of the present disclosure further comprise:
v) a structure-specific nuclease;
wherein the structure-specific nuclease is preferably FEN1.

In some embodiments, the sequence-specific nucleic acid nickase of the present disclosure is a CRISPR-associated protein (Cas) polypeptide having a nickase activity, a zinc finger nuclease (ZFN), a transcription activator-like effector nuclease (TALEN), a meganuclease, a sequence-specific endonuclease, or a variant, fragment and combination thereof.

In some embodiments, the sequence-specific nucleic acid nickase of the present disclosure is the CRISPR-associated protein (Cas) polypeptide having a nickase activity; and the components of the strand displacement-based gene editing system further comprise: vi) a guide RNA that guides a corresponding Cas polypeptide to a target nucleotide region.

In some embodiments, the Cas polypeptide of the present disclosure is selected from Cas3, Cas4, Cas5, Cas5e, Cas6, Cas6e, Cas6f, Cas7, Cas8a1, Cas8a2, Cas8b, Cas8c, Cas9, Cas10, Cas10d, Cas12, Cas13, Cas14, CasX, CasF, CasG, CasH, Csy1, Csy2, Csy3, Cse1, Cse2, Cse3, Cse4, Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, TranC, and a variant, fragment or any combination thereof.

In some embodiments, the sequence-specific nucleic acid nickase of the present disclosure comprises a mutant comprising a D10A mutation or an H840A mutation corresponding to the amino acid sequence set forth in SEQ ID NO:1.

In some embodiments, the Twinkle enzyme of the present disclosure is a naturally occurring or truncated Twinkle enzyme; and the Twinkle enzyme comprises a carboxyl-terminal domain (CTD) having an amino acid sequence set forth in SEQ ID NO: 2.

In some embodiments, the exonuclease of the present disclosure is a 3′ exonuclease or a 5′ exonuclease or a combination thereof; wherein the 3′ exonuclease is preferably Trex2; and the 5′ exonuclease is preferably mExol or T5 exonuclease.

In some embodiments, a nuclear localization signal (NLS) is fused to an amino or carboxyl terminus of at least one of the components of the present disclosure. In some embodiments, NLS comprises the amino acid sequence KKRKV (SEQ ID NO: 16), PKKKRKV (SEQ ID NO: 17), KRPAATKKAGQAKKKK (SEQ ID NO: 18), KRTADGSEFESPKKKRKV (SEQ ID NO: 19) or DSLLMNRRKFLYQFKNVRWAKGRRETYLC (SEQ ID NO: 20).

In some embodiments, the linker of the present disclosure comprises an amino acid sequence (GGGS)n (SEQ ID NO: 21), (GGGGS)n (SEQ ID NO: 22), (G)n, (EAAAK)n (SEQ ID NO: 23), (GGS)n, (SGGS)n (SEQ ID NO: 24), or SGSETPGTSESATPES (SEQ ID NO:25), or a (XP)n motif or a combination thereof, wherein n is independently an integer of 1 to 30, and wherein X is any amino acid.

In some embodiments, each of the components of the system of the present disclosure is recruited via a recruitment system that is a MCP-MS2 system or a GCN4-ScFv system.

In another aspect of the present disclosure, there is provided a host cell, wherein the host cell comprises the strand displacement-based gene editing system of the present disclosure.

In another aspect of the present disclosure, there is provided a method for generating at least one cell that is genetically modified, wherein the method comprises editing the at least one cell by the method of the present disclosure, or introducing the strand displacement-based gene editing system of the present disclosure into the at least one cell to cause a substitution, insertion or deletion of one or more nucleotides within a target nucleotide editing region in the at least one cell.

In some embodiments, the method of the present disclosure further comprises a step of screening the at least one cell for a cell having one or more desired nucleotide substitutions.

In some embodiments, the cell of the present disclosure is derived from a prokaryote such as bacteria; or an eukaryote such as plants, fungi or vertebrates.

In some embodiments, the vertebrates of the present disclosure are mammals such as human, mouse, rat, monkey, dog, pig, sheep, cattle, and cat.

In some embodiments, the plants of the present disclosure are crop plants, such as wheat, rice, corn, soybean, sunflower, sorghum, oilseed rape, alfalfa, cotton, barley, millet, sugar cane, tomato, tobacco, cassava or potato.

In another aspect of the present disclosure, there is provided use of the method for performing strand displacement-based gene editing in a DNA sequence according to the present disclosure or the strand displacement-based gene editing system according to the present disclosure, wherein the use comprises:
a) gene or genome editing;
b) detection and/or diagnosis of a target nucleotide;
c) editing of a target nucleotide sequence to modify an organism or a non-human organism; and
d) treatment of a disease.

In another aspect of the present disclosure, there is provided a kit, wherein the kit comprises the strand displacement-based gene editing system of the present disclosure or the host cell of the present disclosure.

The gene editing system and the method for generating a genetically modified cell according to the present disclosure are particularly suitable for genetically modifying plants. Preferably, the plants are crop plants including, but not limited to, wheat, rice, corn, soybean, sunflower, sorghum, oilseed rape, alfalfa, cotton, barley, millet, sugar cane, tomato, tobacco, cassava, and potato. More preferably, the plants are rice.

In another aspect, the present disclosure provides a method for producing a genetically modified plant, comprising introducing the gene editing system of the present disclosure into at least one plant, thereby resulting in one or more nucleotide substitutions within a target nucleic acid region in a genome of the at least one plant.

In some embodiments, the method further comprises screening the at least one plant for a plant with one or more desired nucleotide substitutions.

In the method of the present disclosure, the gene editing composition may be introduced into a plant via various methods well-known to those skilled in the art. The methods usable to introduce the gene editing system of the present disclosure into a plant include, but are not limited to: particle bombardment, PEG-mediated protoplast transformation, Agrobacterium-mediated transformation, plant virus-mediated transformation, pollen tube pathway, and ovary injection. Preferably, the gene editing composition is introduced into a plant via transient transformation.

In the method of the present disclosure, the modification of the target sequence may be achieved by simply introducing or producing the base editing system into a plant cell, and the modification may be stably inherited without the need of the stable transformation of the exogenous polynucleotide encoding the components of the gene editing system into the plant. This avoids the potential off-target effects of the stably existing (continuously produced) gene editing composition, and also avoids the integration of the exogenous nucleotide sequence(s) in the plant genome, thereby exhibiting higher biological safety.

In some preferred embodiments, the introduction is performed in the absence of selection pressure, thereby avoiding the integration of the exogenous nucleotide sequence(s) in the plant genome.

In some embodiments, the introduction includes transforming the gene editing system of the present disclosure into an isolated plant cell or tissue and then enabling the regeneration of the transformed plant cell or tissue into an intact plant. Preferably, the regeneration is performed in the absence of selection pressure, that is, any selective agent for the selector gene carried on the expression vector is not used during tissue culture. The regeneration efficiency of plant may be enhanced without the use of a selective agent, and a modified plant that does not comprise an exogenous nucleotide sequence is obtained.

In other embodiments, the gene editing system of the present disclosure may be transformed into a specific part of an intact plant, such as leaf, stem tip, pollen tube, young ear, or hypocotyl. This is particularly suitable for the transformation of the plants that are difficult to regenerate by tissue culture.

In some embodiments of the present disclosure, the protein expressed in vitro and/or the RNA molecule transcribed in vitro (e.g. the expression construct is an RNA molecule transcribed in vitro) are directly transformed into the plant. The protein and/or RNA molecule are capable of achieving gene editing in plant cells and subsequently degraded by cells, avoiding the integration of exogenous nucleotide sequence in the plant genome.

Therefore, in some embodiments, a plant whose genome is free of the integrated exogenous polynucleotide, i.e., a transgene-free modified plant, may be obtained by conducting the genetic modification and breeding of plant using the method of the present disclosure.

In some embodiments of the present disclosure, the modified target nucleic acid region is associated with plant traits such as an agronomic trait. As a result, the one or more nucleotide substitutions result in the plant having altered (preferably, improved) traits such as an agronomic trait, as compared to the wild-type plant.

In some embodiments, the method further comprises a step of screening a plant having one or more nucleotide substitutions as desired and/or a desired trait such as an agronomic trait.

In some embodiments of the present disclosure, the method further comprises obtaining the progeny of the genetically modified plant. Preferably, the genetically modified plant or the progeny thereof has one or more nucleotide substitutions as desired and/or a desired trait such as an agronomic trait.

In another aspect, the present disclosure further provides a genetically modified plant, a progeny thereof or a part thereof, wherein the plant is obtained by the above-mentioned method of the present disclosure. In some embodiments, the genetically modified plant, the progeny thereof or the part thereof is non-transgenic. Preferably, the genetically modified plant or the progeny thereof has a desired genetic modification and/or a desired trait such as an agronomic trait.

In another aspect, the present disclosure further provides a method for plant breeding, comprising hybridizing a first genetically modified plant that comprises one or more nucleotide substitutions in the target nucleic acid region and is obtained by the above-mentioned method of the present disclosure with a second plant free of the one or more nucleotide substitutions, thereby introducing the one or more nucleotide substitutions into the second plant. Preferably, the first genetically modified plant has a desired trait such as an agronomic trait.

The present disclosure further encompasses use of the gene editing system of the present disclosure in treatment of a disease.

The up-regulation, down-regulation, inactivation, activation, mutation correction or the like of disease-related genes may be achieved by modifying the disease-related genes with the gene editing system of the present disclosure, thereby realizing the prevention and/or treatment of diseases. For example, the target nucleic acid region according to the present disclosure may be located in the protein coding region of a disease-related gene or, for example, may be located in a gene expression regulatory region such as a promoter region or an enhancer region, thereby enabling the functional modification of the disease-related gene or the modification of the expression of the disease-related gene. Therefore, the modifications of a disease-related gene described herein include not only the modifications of the disease-related gene itself (for example, the protein coding region), but also the modifications of its expression regulatory regions (such as a promoter, an enhancer, and an intron).

A “disease-related” gene refers to any gene that produces a transcription or translation product at an abnormal level or in an abnormal form in cells derived from a disease-affected tissue as compared to the non-disease control tissue or cell. In a case where the modified expression is associated with the occurrence and/or progression of a disease, it may be a gene expressed at an abnormally high level; and it may be a gene expressed at an abnormally low level. A disease-related gene also refers to a genetically mutated gene that has one or more mutations, or is directly responsible for the etiology of the disease or in linkage disequilibrium with one or more genes responsible for the etiology of the disease. The mutation or genetic variation is, for example, a single nucleotide variation (SNV). The transcription or translation product may be known or unknown, and may be at a normal or abnormal level.

Accordingly, the present disclosure further provides a method for treating a disease in a subject in need thereof, comprising delivering an effective amount of the gene editing system of the present disclosure to the subject so as to modify a gene related to the disease. The present disclosure further provides use of the gene editing system in preparation of a pharmaceutical composition for treating a disease in a subject in need thereof, wherein the gene editing system is used to modify a gene related to the disease. The present disclosure further provides a pharmaceutical composition for treating a disease in a subject in need thereof, comprising the gene editing system of the present disclosure and optionally a pharmaceutically acceptable carrier, wherein the gene editing system is used to modify a gene related to the disease.

In some embodiments, the gene editing system described in the present disclosure is used to introduce a point mutation into a nucleic acid. In some embodiments, the mutation results in the correction of a genetic defect, for example, upon correcting a point mutation that leads to the loss of function in the genetic product. In some embodiments, the genetic defect is associated with a disease or condition (for example, lysosomal storage disease or a metabolic disease such as Type I diabetes). In some embodiments, the method provided herein may be used to introduce an inactivating point mutation into a gene or allele encoding a genetic product associated with the disease or condition.

In some embodiments, the embodiments described in the present disclosure are intended to restore the function of a dysfunctional gene via genome editing. The nuclear gene editing protein provided herein may be used for in-vitro gene editing of human cells, such as the correction of a disease-related mutation in a human cell culture. The nuclear gene editing protein provided herein may be used to correct any single point mutation from G to A or from C to T.

In some embodiments, the embodiments described in the present disclosure are intended to treat a disease associated with or caused by a point mutation, and the point mutation may be corrected by the DNA gene editing system provided herein. In some embodiments, the disease is a proliferative disease. In some embodiments, the disease is a genetic disease. In some embodiments, the disease is a de novo disease. In some embodiments, the disease is a metabolic disease. In some embodiments, the disease is lysosomal storage disease.

In some embodiments, the embodiments described in the present disclosure are intended to treat mitochondrial diseases or disorders. As used herein, a “mitochondrial disease” refers to a disease caused by abnormal mitochondria, for example, a mitochondrial gene mutation or a gene mutation in enzymatic pathway. Examples of the disease include, but are not limited to: neurological diseases, loss of motion control, muscle weakness and pain, gastrointestinal diseases and difficulty in swallowing, poor growth, heart diseases, liver diseases, diabetes, respiratory complications, epilepsy, vision/hearing problems, lactic acidosis, developmental retardation and susceptibility to infection.

Examples of the diseases according to the present disclosure include, but are not limited to, genetic diseases, circulatory system diseases, muscle diseases, diseases of brain and the central nervous and immune systems, Alzheimer's disease, secretase disorders, amyotrophic lateral sclerosis (ALS), autism, trinucleotide repeat expansion disorders, hearing diseases, gene-targeted therapy of non-dividing cells (neurons and muscle cells), liver and kidney diseases, diseases in epithelial cells and lung, cancer, Usher syndrome or retinitis pigmentosa, cystic fibrosis, HIV and AIDS, β-mediterranean anemia, sickle cell disease, herpes simplex virus, autistic, drug addiction, age-related macular degeneration, and schizophrenia. Other diseases that can be treated via the point mutation correction or the introduction of an inactivating mutation into a disease-related gene are also known to those skilled in the art, and therefore the present disclosure is not limited to the diseases listed herein. The strategies and the fusion proteins provided in the present disclosure may also be used for treatment of other related diseases, in addition to the diseases exemplarily described herein, and this application is apparent to those skilled in the art. Reference for diseases or targets applicable to the present disclosure can be found in the following documents, which describe the related diseases to which the gene editing system is applicable: WO2015089465A1 (PCT/US2014/070135), WO2016205711A1 (PCT/US2016/038181), WO2018141835A1 (PCT/EP2018/052491), WO2020191234A1 (PCT/US2020/023713), WO2020191233A1 (PCT/US2020/023712), WO2019079347A1 (PCT/US2018/056146), and WO2021155065A1 (PCT/US2021/015580).

Administration of the gene editing system or pharmaceutical composition of the present disclosure may be tailored to the body weight and species of the patient or subject. The frequency of administration is within the medically or veterinarily permissible scope. It depends on conventional factors including the age, gender, general health status, and other conditions of the patient or subject, and the particular condition or symptoms to be treated.

The present disclosure further comprises a kit for use in the method of the present disclosure, the kit comprising the gene editing system of the present disclosure and instructions for use. The kit generally contains a label indicating the intended use and/or usage method for the contents of the kit. The term “label” includes any written or recorded material provided on or with the kit or otherwise provided with the kit.

### Definitions

In the present disclosure, the scientific and technical terms used herein have the meaning as commonly understood by those skilled in the art unless otherwise specified. Also, the terms related to the protein and nucleic acid chemistry, molecular biology, cell and tissue culture, microbiology, and immunology as well as the laboratory procedures described herein are terms and conventional steps widely used in the pertinent fields. For example, standard recombinant DNA and molecular cloning techniques employed in the present disclosure are well known to those skilled in the art and are described in greater details in the following literature: Sambrook, J., Fritsch, E. F. and Maniatis, T., Molecular Cloning: A Laboratory Manual; Cold Spring Harbor Laboratory Press: Cold Spring Harbor, 1989 (hereinafter referred to as “Sambrook”). Meanwhile, to facilitate understanding of the present disclosure, definitions and explanations of the relevant terms are described below.

The “genome”, as used herein, encompasses not only chromosomal DNA present in the nucleus, but also organellar DNA present in the subcellular components (e.g. mitochondria and plastids) of the cell.

As used herein, the “organism” includes any organism, preferably eukaryotic organism, suitable for genome editing. Examples of the organism include, but are not limited to, mammals such as humans, mice, rats, monkeys, dogs, pigs, sheep, cattle, and cats; poultry such as chickens, ducks, and geese; and plants including monocots and dicots, such as rice, corn, wheat, sorghum, barley, soybean, peanut, and arabidopsis.

The “genetically modified organism” or “genetically modified cell” means an organism or cell containing, within its genome, an exogenous polynucleotide or a modified gene or expression regulatory sequence. For example, the exogenous polynucleotide may be stably integrated into the genome of the organism or cell and inherited through successive generations. The exogenous polynucleotide may be integrated into the genome individually or as part of a recombinant DNA construct. The modified gene or expression regulatory sequence means that the sequence comprises one or more nucleotide substitutions, deletions and additions in the organism or cell genome.

The “exogenous” in reference to a sequence means a sequence that originates from a foreign species or, if originating from the same species, means a sequence whose composition and/or locus has been substantially altered from its native form by means of deliberate human intervention.

The “polynucleotide”, “nucleic acid sequence”, “nucleotide sequence”, and “nucleic acid fragment” may be used interchangeably to denote a single-stranded or double-stranded RNA or DNA polymer, which may optionally contain synthetic, non-natural or altered nucleotide bases. Nucleotides are designated by their single-letter names as follows: “A” denotes adenosine or deoxyadenosine (corresponding to RNA or DNA, respectively), “C” denotes cytidine or deoxycytidine, “G” denotes guanosine or deoxyguanosine, “U” denotes uridine, “T” denotes deoxythymidine, “R” denotes purine (A or G), “Y” denotes pyrimidine (C or T), “K” denotes G or T, “H” denotes A or C or T, “I” denotes inosine, and “N” denotes any nucleotide.

The “polypeptide”, “peptide”, and “protein” are used interchangeably in the present disclosure to denote a polymer of amino acid residues. These terms apply to amino acid polymers in which one or more amino acid residues are artificial chemical analogue(s) of the corresponding naturally occurring amino acid(s) and to naturally occurring amino acid polymers. The terms “polypeptide”, “peptide,” “amino acid sequence”, and “protein” may also include modified forms thereof, including but not limited to: glycosylation, lipid ligation, sulfation, γ-carboxylation of glutamic acid residues, hydroxylation, and ADP-ribosylation.

As used in the present disclosure, the “expression construct” refers to a vector (such as a recombinant vector) suitable for expression of a nucleotide sequence of interest in an organism. The “expression” refers to production of a functional product. For example, the expression of a nucleotide sequence may refer to the transcription of a nucleotide sequence (e.g. transcription to produce mRNA or functional RNA) and/or the translation of RNA into a precursor or a mature protein.

The “expression construct” of the present disclosure may be a linear nucleic acid fragment, a circular plasmid, a viral vector or, in some embodiments, a translatable RNA (such as mRNA).

The “expression construct” of the present disclosure may include regulatory sequences and nucleotide sequences of interest that are derived from different origins, or regulatory sequences and nucleotide sequences of interest that, while derived from the same origin, are arranged in a different manner from the conventional native one.

### EXAMPLES

For ease of understanding, the present disclosure will be described in greater detail below with reference to the specific embodiments and appended drawings. The appended drawings illustrate the preferred embodiments of the present disclosure. However, the present disclosure may be implemented in various different forms and is not limited to the examples described herein. These examples are provided to ensure a clearer and more comprehensive understanding of the present disclosure.

### Example 1: Acquisition of Strand Displacement-Based Gene Editing System (Twinkle Editor)

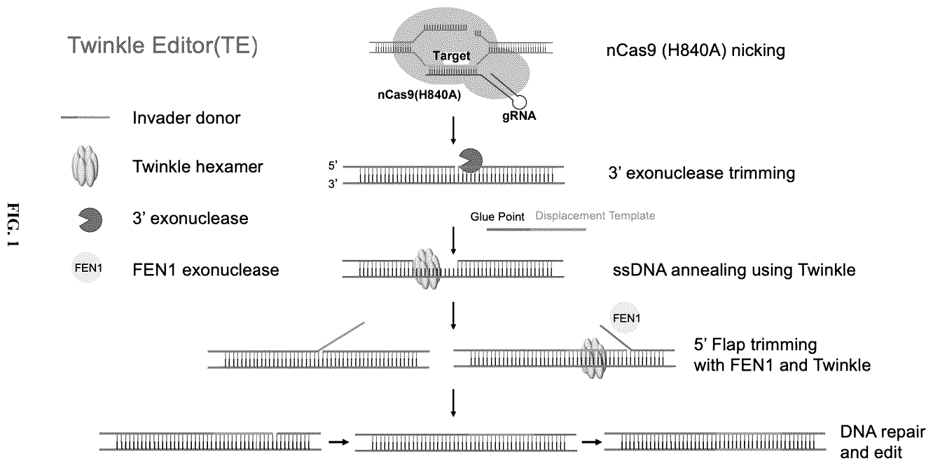

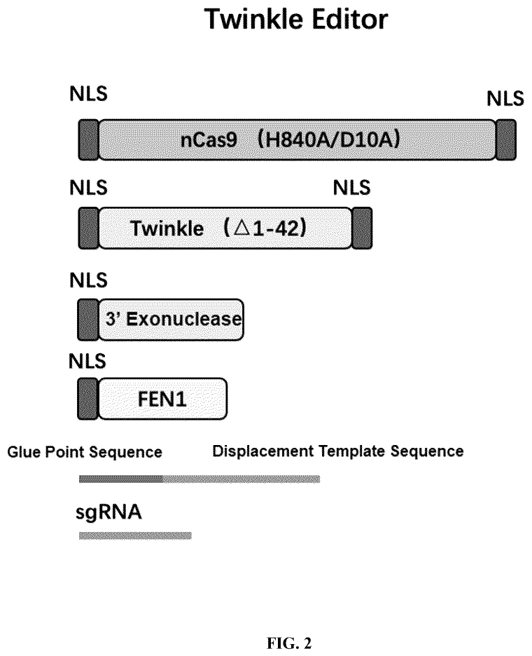

The present inventors constructed a Twinkle Editor comprising the following protein components: (1) a nickase nCas9: the Cas9 (D10A/H840A) protein (SEQ ID NO: 3 or 4); (2) a human Twinkle protein with 42 amino acids (encoding one mitochondrial localization signal) removed from the amino terminus (SEQ ID NO: 2); (3) exonucleases (SEQ ID NOs: 5-7); and (4) an FEN1 protein (SEQ ID NO: 8). One or two nuclear localization signal NLS sequence(s) were fused to the terminus/termini of each of the protein components. The coding nucleotide sequence of each of the protein components was cloned into the pCMV vector suitable for expression in human cells and used for protein expression. Cas9 (H840A) was guided by sgRNA to the target DNA, and an appropriate invader DNA was synthesized in vitro according to the desired editing type. The invader DNA included a glue point sequence complementary to the free single-stranded DNA of the target DNA and a displacement template sequence containing a desired nucleotide alteration. The above components constituted the strand displacement-based gene editing system (Twinkle Editor) according to the present disclosure. A schematic diagram for the structure of an exemplary Twinkle Editor in a free state was shown in FIG. 2 and its working principle was shown in FIG. 1.

### Example 2: Genome Editing in Human Cells

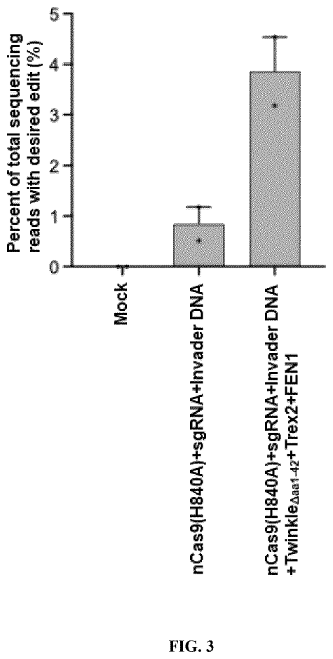

Employed was the gene editing system obtained in Example 1, comprising the following protein components: (1) a Cas9 (H840A) protein (SEQ ID NO: 4); (2) a human Twinkle protein with 42 amino acids (encoding one mitochondrial localization signal) removed from the amino terminus (SEQ ID NO: 2); (3) a 3′ exonuclease Trex2 (SEQ ID NO: 5); and (4) an FEN1 protein (SEQ ID NO: 8). Among them, a NLS sequence was fused to both the N and C termini of the nCas9 protein and the Twinkle protein, and a NLS sequence was fused to the N termini of Trex2 and FEN1. An invader DNA sequence was designed for editing the human HEK3 gene (HGNC ID: 10747). The sgRNA targeted the sequence GGCCCAGACTGAGCACGTGATGG (SEQ ID NO: 26). The invader DNA sequence designed for T-A base displacement was gagcacgAGATGGCAAAGGAAAGGAAGCCCTGC (SEQ ID NO: 27), where the lowercase letters denoted the nucleotide sequence of the glue point of the invader DNA, the uppercase letters denoted the nucleotide sequence of the displacement template, and the bold letter denoted the target editing nucleotide sequence to be introduced. The plasmids (pCMV-NLS-Cas9 (H840A)-NLS; pCMV-NLS-hTwinkle_{Δ1-42aa}-NLS; pCMV-NLS-Trex2-NLS; pCMV-NLS-FEN1-NLS) encoding the respective components of the gene editing system, the sgRNA, and the invader DNA were transfected into the HEK293T cell. The system containing only nCas9 and the template sequence of invader DNA was selected as the control group of the nickase system. DNA was extracted 72 hours after transfection. The target site was analyzed by the next-generation sequencing technology and it was found that the desired gene editing event occurred at the target site, suggesting that the gene editing system of the present disclosure could achieve effective gene editing (FIG. 3).

### Example 3: Recruiting Twinkle Protein by Introducing MS2-MCP for Genome Editing in Human Cells

This example further optimized the Twinkle system in Example 2 by introducing an MS2-MCP recruitment system to recruit the components of the Twinkle system at the target site. Its differences from the Twinkle Editor of Example 2 were fusion of an MCP protein between Twinkle and its N-terminal NLS sequence, and introduction of two MS2 aptamer RNAs into sgRNA. The sgRNA guided Cas9 (H840A) to the target DNA, and recruited the MCP protein-fused Twinkle protein through MS2 thereon.

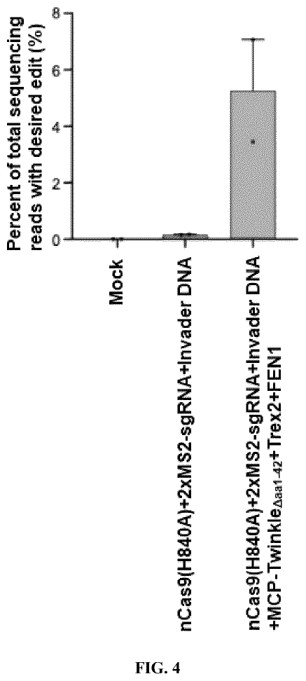

An invader DNA sequence intended for DNA insertion was designed to edit the human RNF2 gene (HGNC ID: 10061). The target sequence of sgRNA was GTCATCTTAGTCATTACCTGAGG. The designed invader DNA sequence was gtcatcttagtcattaccCCTTGAGGTGTTCGTTGTAACTCATATAAACTGAGTTCCCATGTT (SEQ ID NO: 28), where the lowercase letter denoted the nucleotide sequence of the glue point of the invader DNA, the uppercase letters denoted the nucleotide sequence of the displacement template, and the bold letters denoted the target editing nucleotide sequence to be introduced. The plasmid encoding each of the components of the gene editing system and the invader DNA were transfected into the HEK293T cell. The system containing nCas9 and the template sequence of invader DNA, which was recruited by MS2-MCP, was selected as the control group of the nickase system. DNA was extracted 72 hours after transfection. The target site was analyzed by the next-generation sequencing technology and it was found that the desired gene editing event occurred at the target site, that is, successful insertion of the three bases CCT at the target site, which suggested that the gene editing system of the present disclosure could achieve effective gene editing (FIG. 4).

### Example 4: Use of Double-Stranded DNA as Invader DNA for Genome Editing in Human Cells

In this example, the invader DNA was further optimized. In this example, the present inventors tested the Twinkle Editor using a double-stranded DNA as the invader DNA. The difference of this Twinkle Editor from the Twinkle Editor recruited by MS2-MCP in Example 3 was that the invader DNA used was a double-stranded invader DNA sequence obtained through PCR amplification.

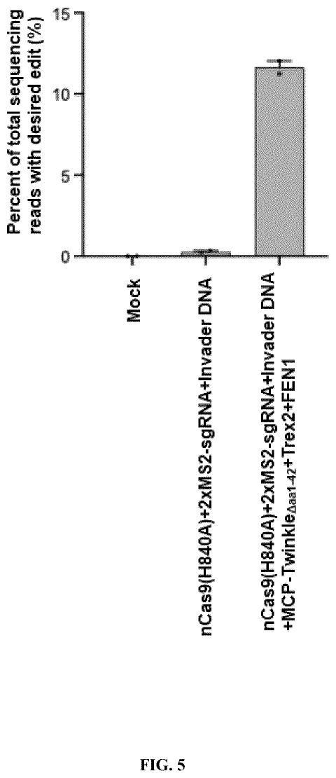

This example was designed for base-deletion editing on the human RUNX1 gene (HGNC ID: 10471). The target sequence was GCATTTTCAGGAGGAAGCGATGG (SEQ ID NO: 29), where the bold letter denoted the sequence intended to be deleted by the Twinkle Editor. The forward sequence of the amplified double-stranded invader DNA was CTAGAGGGGTGAGGCTGAAACAGTGACCTGTCTTGGTTTTCGCTCCGAAGGTAAAA GAAATCATTGAGTCCCCCGCCTTCAGAAGAGGGTGCATTTTCAGGAGGAAGGATGG CTTCAGACAGCATATTTGAGTCATTTCCTTCGTACCCACAGTGCTTCATGAGAGGTG AGTACATGCTGGTCTTGTAATATCTACTTTTGCTCAGCTTTGCCTGTAATGAAATGGC AGCTTGTTTCACCTCGGTGCAGAGATGCCTCGGTGCCTGCCAGTTCCCTGTCTTGTT TGT ID NO: (SEQ 30), the complementary sequence was and ACAAACAAGACAGGGAACTGGCAGGCACCGAGGCATCTCTGCACCGAGGTGAAAC AAGCTGCCATTTCATTACAGGCAAAGCTGAGCAAAAGTAGATATTACAAGACCAGC ATGTACTCACCTCTCATGAAGCACTGTGGGTACGAAGGAAATGACTCAAATATGCTG TCTGAAGCCATCCTTCCTCCTGAAAATGCACCCTCTTCTGAAGGCGGGGGACTCAA TGATTTCTTTTACCTTCGGAGCGAAAACCAAGACAGGTCACTGTTTCAGCCTCACCC CTCTAG (SEQ ID NO: 31). The plasmid encoding each of the components of the gene editing system and the invader DNA were transfected into the HEK293T cell. DNA was extracted 72 hours after transfection. The target site was analyzed by the next-generation sequencing technology and it was found that the desired gene editing event occurred at the target site, that is, the successful deletion of the target base C at the target site, which suggested that the gene editing system of the present disclosure could achieve effective gene editing (FIG. 5).

Although the examples of the present disclosure have been described above, it will be appreciated that the above descriptions are merely exemplary, but not exhaustive; and that the disclosed examples are not limiting. A number of variations and modifications may occur to one skilled in the art without departing from the scopes and spirits of the described examples. The terms in the present disclosure are selected to provide the best explanation on the principles and practical applications of the embodiments and the technical improvements to the arts on market, or to make the examples described herein understandable to one skilled in the art.

Sequence Listing:
Cas9
>SEQ ID NO: 1
MDKKYSIGLDIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNG
RDMYVDQELDINRLSDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGD
Twinkle-CTD (Δ1-42)
>SEQ ID NO: 2
LPAWHKSIVSFRQLREEVLGELSNVEQAAGLRWSRFPDLNRILKGHRKGELTVFTGPTGSGKT
TFISEYALDLCSQGVNTLWGSFEISNVRLARVMLTQFAEGRLEDQLDKYDHWADRFEDLPLYF
MTFHGQQSIRTVIDTMQHAVYVYDICHVIIDNLQFMMGHEQLSTDRIAAQDYIIGVFRKFATD
NNCHVTLVIHPRKEDDDKELQTASIFGSAKASQEADNVLILQDRKLVTGPGKRYLQVSKNRFD
GDVGVFPLEFNKNSLTFSIPPKNKARLKKIKDDTGPVAKKPSSGKKGATTQNSEICSGQAPTP
DQPDTSKRSK
nCas9 (D10A)
>SEQ ID NO: 3
MDKKYSIGLAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNG
RDMYVDQELDINRLSDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGD
nCas9 (H840A)
>SEQ ID NO: 4
MDKKYSIGLDIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNG
RDMYVDQELDINRLSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGD
Trex2
>SEQ ID NO: 5
MSEPPRAETFVFLDLEATGLPNMDPEIAEISLFAVHRSSLENPERDDSGSLVLPRVLDKLTLC
MCPERPFTAKASEITGLSSESLMHCGKAGFNGAVVRTLQGFLSRQEGPICLVAHNGFDYDFPL
LCTELQRLGAHLPQDTVCLDTLPALRGLDRAHSHGTRAQGRKSYSLASLFHRYFQAEPSAAHS
AEGDVHTLLLIFLHRAPELLAWADEQARSWAHIEPMYVPPDGPSLEA
mExoI
>SEQ ID NO: 6
MGIQGLLQFIQEASEPVNVKKYKGQAVAVDTYCWLHKGAIACAEKLAKGEPTDRYVGFCMKFV
NMLLSYGVKPILIFDGCTLPSKKEVERSRRERRQSNLLKGKQLLREGKVSEARDCFARSINIT
HAMAHKVIKAARALGVDCLVAPYEADAQLAYLNKAGIVQAVITEDSDLLAFGCKKVILKMDQF
GNGLEVDQARLGMCKQLGDVFTEEKFRYMCILSGCDYLASLRGIGLAKACKVLRLANNPDIVK
VIKKIGHYLRMNITVPEDYITGFIRANNTFLYQLVFDPIQRKLVPLNAYGDDVNPETLTYAGQ
YVGDSVALQIALGNRDVNTFEQIDDYSPDTMPAHSRSHSWNEKAGQKPPGTNSIWHKNYCPRL
EVNSVSHAPQLKEKPSTLGLKQVISTKGLNLPRKSCVLKRPRNEALAEDDLLSQYSSVSKKIK
ENGCGDGTSPNSSKMSKSCPDSGTAHKTDAHTPSKMRNKFATFLQRRNEESGAVVVPGTRSRF
FCSSQDFDNFIPKKESGQPLNETVATGKATTSLLGALDCPDTEGHKPVDANGTHNLSSQIPGN
AAVSPEDEAQSSETSKLLGAMSPPSLGTLRSCFSWSGTLREFSRTPSPSASTTLQQFRRKSDP
PACLPEASAVVTDRCDSKSEMLGETSQPLHELGCSSRSQESMDSSCGLNTSSLSQPSSRDSGS
EESDCNNKSLDNQGEQNSKQHLPHFSKKDGLRRNKVPGLCRSSSMDSFSTTKIKPLVPARVSG
LSKKSGSMQTRKHHDVENKPGLQTKISELWKNFGFKKDSEKLPSCKKPLSPVKDNIQLTPETE
DEIFNKPECVRAQRAIFH
T5 Exonuclease
>SEQ ID NO: 7
MSKSWGKFIEEEEAEMASRRNLMIVDGTNLGFRFKHNNSKKPFASSYVSTIQSLAKSYSARTT
IVLGDKGKSVFRLEHLPEYKGNRDEKYAQRTEEEKALDEQFFEYLKDAFELCKTTFPTFTIRG
VEADDMAAYIVKLIGHLYDHVWLISTDGDWDTLLTDKVSRFSFTTRREYHLRDMYEHHNVDDV
EQFISLKAIMGDLGDNIRGVEGIGAKRGYNIIREFGNVLDIIDQLPLPGKQKYIQNLNASEEL
LFRNLILVDLPTYCVDAIAAVGQDVLDKFTKDILEIAEQ
FEN1
>SEQ ID NO: 8
MGIQGLAKLIADVAPSAIRENDIKSYFGRKVAIDASMSIYQFLIAVRQGGDVLQNEEGETTSH
LMGMFYRTIRMMENGIKPVYVFDGKPPQLKSGELAKRSERRAEAEKQLQQAQAAGAEQEVEKF
TKRLVKVTKQHNDECKHLLSLMGIPYLDAPSEAEASCAALVKAGKVYAAATEDMDCLTFGSPV
LMRHLTASEAKKLPIQEFHLSRILQELGLNQEQFVDLCILLGSDYCESIRGIGPKRAVDLIQK
HKSIEEIVRRLDPNKYPVPENWLHKEAHQLFLEPEVLDPESVELKWSEPNEEELIKFMCGEKQ
FSEERIRSGVKRLSKSRQGSTQGRLDDFFKVTGSLSSAKRKEPEPKGSTKKKAKTGAAGKFKR
GK*
NLS
>SEQ ID NO: 9
MKRTADGSEFESPKKKRKV
MS2 Capsid Protein (MCP)
>SEQ ID NO: 10
MASNFTQFVLVDNGGTGDVTVAPSNFANGIAEWISSNSRSQAYKVTCSVRQSSAQNRKYTIKV
EVPKGAWRSYLNMELTIPIFATNSDCELIVKAMQGLLKDGNPIPSAIAANSGIY
MS2
>SEQ ID NO: 11
ACATGAGGATCACCCATGT
pCMV-NLS-Cas9(H840A)-NLS Plasmid Sequence:
>SEQ ID NO: 12
GAAGATCCTTTGATCTTTTCTACGGGGTCTGACACTCAGTGGAACGAAAACTCACGTTAAGGG
ATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGTGTCA
CGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCG
CGCACATTTCCCCGAAAAGTGCCACCTGACGTCGACGGATCGGGAGATCGATCTCCCGATCCC
CTAGGGTCGACTCTCAGTACAATCTGCTCTGATGCCGCATAGTTAAGCCAGTATCTGCTCCCT
GCTTGTGTGTTGGAGGTCGCTGAGTAGTGCGCGAGCAAAATTTAAGCTACAACAAGGCAAGGC
TTGACCGACAATTGCATGAAGAATCTGCTTAGGGTTAGGCGTTTTGCGCTGCTTCGCGATGTA
CGGGCCAGATATACGCGTTGACATTGATTATTGACTAGTTATTAATAGTAATCAATTACGGGG
TCATTAGTTCATAGCCCATATATTGAGTTCCGCGTTACATAACTTACGGTAAATGGCCCGCCT
GGCTGACCGCCCAACGACCCCCGCCCATTGACGTCAATAATGACGTATGTTCCCATAGTAACG
CCAATAGGGACTTTCCATTGACGTCAATGGGTGGAGTATTTACGGTAAACTGCCCACTTGGCA
GTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTA
TTAGTCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATGGGCGGT
AGGCGTGTACGGTGGGAGGTCTATATAAGCAGAGCTGGTTTAGTGAACCGTCAGATCCGCTAG
AGATCCGCGGCCGCATGAAAAGAACCGCTGATGGCAGCGAGTTCGAGAGCCCTAAGAAGAAGC
GGAAGGTGATGgacaagaagtacagcatcggcctggacatcggcaccaactctgtgggctggg
ccgtgatcaccgacgagtacaaggtgcccagcaagaaattcaaggtgctgggcaacaccgacc
ggcacagcatcaagaagaacctgatcggagccctgctgttcgacagcggcgaaacagccgagg
ccacccggctgaagagaaccgccagaagaagatacaccagacggaagaaccggatctgctatc
tgcaagagatcttcagcaacgagatggccaaggtggacgacagcttcttccacagactggaag
agtccttcctggtggaagaggataagaagcacgagcggcaccccatcttcggcaacatcgtgg
acgaggtggcctaccacgagaagtaccccaccatctaccacctgagaaagaaactggtggaca
gcaccgacaaggccgacctgcggctgatctatctggccctggcccacatgatcaagttccggg
gccacttcctgatcgagggcgacctgaaccccgacaacagcgacgtggacaagctgttcatcc
agctggtgcagacctacaaccagctgttcgaggaaaaccccatcaacgccagcggcgtggacg
ccaaggccatcctgtctgccagactgagcaagagcagacggctggaaaatctgatcgcccagc
tgcccggcgagaagaagaatggcctgttcggaaacctgattgccctgagcctgggcctgaccc
ccaacttcaagagcaacttcgacctggccgaggatgccaaactgcagctgagcaaggacacct
acgacgacgacctggacaacctgctggcccagatcggcgaccagtacgccgacctgtttctgg
ccgccaagaacctgtccgacgccatcctgctgagcgacatcctgagagtgaacaccgagatca
ccaaggcccccctgagcgcctctatgatcaagagatacgacgagcaccaccaggacctgaccc
tgctgaaagctctcgtgcggcagcagctgcctgagaagtacaaagagattttcttcgaccaga
gcaagaacggctacgccggctacattgacggcggagccagccaggaagagttctacaagttca
tcaagcccatcctggaaaagatggacggcaccgaggaactgctcgtgaagctgaacagagagg
acctgctgcggaagcagcggaccttcgacaacggcagcatcccccaccagatccacctgggag
agctgcacgccattctgcggcggcaggaagatttttacccattcctgaaggacaaccgggaaa
agatcgagaagatcctgaccttccgcatcccctactacgtgggccctctggccaggggaaaca
gcagattcgcctggatgaccagaaagagcgaggaaaccatcaccccctggaacttcgaggaag
tggtggacaagggcgcttccgcccagagcttcatcgagcggatgaccaacttcgataagaacc
tgcccaacgagaaggtgctgcccaagcacagcctgctgtacgagtacttcaccgtgtataacg
agctgaccaaagtgaaatacgtgaccgagggaatgagaaagcccgccttcctgagcggcgagc
agaaaaaggccatcgtggacctgctgttcaagaccaaccggaaagtgaccgtgaagcagctga
aagaggactacttcaagaaaatcgagtgcttcgactccgtggaaatctccggcgtggaagatc
ggttcaacgcctccctgggcacataccacgatctgctgaaaattatcaaggacaaggacttcc
tggacaatgaggaaaacgaggacattctggaagatatcgtgctgaccctgacactgtttgagg
acagagagatgatcgaggaacggctgaaaacctatgcccacctgttcgacgacaaagtgatga
agcagctgaagcggcggagatacaccggctggggcaggctgagccggaagctgatcaacggca
tccgggacaagcagtccggcaagacaatcctggatttcctgaagtccgacggcttcgccaaca
gaaacttcatgcagctgatccacgacgacagcctgacctttaaagaggacatccagaaagccc
aggtgtccggccagggcgatagcctgcacgagcacattgccaatctggccggcagccccgcca
ttaagaagggcatcctgcagacagtgaaggtggtggacgagctcgtgaaagtgatgggccggc
acaagcccgagaacatcgtgatcgaaatggccagagagaaccagaccacccagaagggacaga
agaacagccgcgagagaatgaagcggatcgaagagggcatcaaagagctgggcagccagatcc
tgaaagaacaccccgtggaaaacacccagctgcagaacgagaagctgtacctgtactacctgc
agaatggggggatatgtacgtggaccaggaactggacatcaaccggctgtccgactacgatgt
ggacgctatcgtgcctcagagctttctgaaggacgactccatcgacaacaaggtgctgaccag
aagcgacaagaaccggggcaagagcgacaacgtgccctccgaagaggtcgtgaagaagatgaa
gaactactggcggcagctgctgaacgccaagctgattacccagagaaagttcgacaatctgac
caaggccgagagaggcggcctgagcgaactggataaggccggcttcatcaagagacagctggt
ggaaacccggcagatcacaaagcacgtggcacagatcctggactcccggatgaacactaagta
cgacgagaatgacaagctgatccgggaagtgaaagtgatcaccctgaagtccaagctggtgtc
cgatttccggaaggatttccagttttacaaagtgcgcgagatcaacaactaccaccacgccca
cgacgcctacctgaacgccgtcgtgggaaccgccctgatcaaaaagtaccctaagctggaaag
cgagttcgtgtacggcgactacaaggtgtacgacgtgcggaagatgatcgccaagagcgagca
ggaaatcggcaaggctaccgccaagtacttcttctacagcaacatcatgaactttttcaagac
cgagattaccctggccaacggcgagatccggaagcggcctctgatcgagacaaacggcgaaac
cggggagatcgtgtgggataagggccgggattttgccaccgtgcggaaagtgctgagcatgcc
ccaagtgaatatcgtgaaaaagaccgaggtgcagacaggcggcttcagcaaagagtctatcct
gcccaagaggaacagcgataagctgatcgccagaaagaaggactgggaccctaagaagtacgg
cggcttcgacagccccaccgtggcctattctgtgctggtggtggccaaagtggaaaagggcaa
gtccaagaaactgaagagtgtgaaagagctgctggggatcaccatcatggaaagaagcagctt
cgagaagaatcccatcgactttctggaagccaagggctacaaagaagtgaaaaaggacctgat
catcaagctgcctaagtactccctgttcgagctggaaaacggccggaagagaatgctggcctc
tgccggcgaactgcagaagggaaacgaactggccctgccctccaaatatgtgaacttcctgta
cctggccagccactatgagaagctgaagggctcccccgaggataatgagcagaaacagctgtt
tgtggaacagcacaagcactacctggacgagatcatcgagcagatcagcgagttctccaagag
agtgatcctggccgacgctaatctggacaaagtgctgtccgcctacaacaagcaccgggataa
gcccatcagagagcaggccgagaatatcatccacctgtttaccctgaccaatctgggagcccc
tgccgccttcaagtactttgacaccaccatcgaccggaagaggtacaccagcaccaaagaggt
gctggacgccaccctgatccaccagagcatcaccggcctgtacgagacacggatcgacctgtc
tcagctgggaggtgacAGCGGCGGCAGCAAAAGAACCGCCGACGGCTCAGAGTTCGAGCCCAA
AAAGAAGCGGAAAGTGTAAGTTTAAACCCGCTGATCAGCCTCGACTGTGCCTTCTAGTTGCCA
GCCATCTGTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGA
TGCGGTGGGCTCTATGGCTTCTGAGGCGGAAAGAACCAGCTGGGGCTCGATACCGTCGACCTC
TAGCTAGAGCTTGGCGTAATCATGGTCATAGCTGTTTCCTGTGTGAAATTGTTATCCGCTCAC
AATTCCACACAACATACGAGCCGGAAGCATAAAGTGTAAAGCCTAGGGTGCCTAATGAGTGAG
CTAACTCACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCTCTTCCGC
TTCCTCGCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGTGAGCAAA
AGGCCAGCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCGG
TGCTACAGAGTTCTTGAAGTGGTGGCCTAACTACGGCTACACTAGAAGAACAGTATTTGGTAT
CTGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAGG
ATCTCAA
pCMV-NLS-hTwinkleΔ1-42aa-NLS Plasmid Sequence:
>SEQ ID NO: 13
GAAGATCCTTTGATCTTTTCTACGGGGTCTGACACTCAGTGGAACGAAAACTCACGTTAAGGG
ATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGTGTCA
CGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCG
CGCACATTTCCCCGAAAAGTGCCACCTGACGTCGACGGATCGGGAGATCGATCTCCCGATCCC
CTAGGGTCGACTCTCAGTACAATCTGCTCTGATGCCGCATAGTTAAGCCAGTATCTGCTCCCT
GCTTGTGTGTTGGAGGTCGCTGAGTAGTGCGCGAGCAAAATTTAAGCTACAACAAGGCAAGGC
TTGACCGACAATTGCATGAAGAATCTGCTTAGGGTTAGGCGTTTTGCGCTGCTTCGCGATGTA
CGGGCCAGATATACGCGTTGACATTGATTATTGACTAGTTATTAATAGTAATCAATTACGGGG
TCATTAGTTCATAGCCCATATATTGAGTTCCGCGTTACATAACTTACGGTAAATGGCCCGCCT
GGCTGACCGCCCAACGACCCCCGCCCATTGACGTCAATAATGACGTATGTTCCCATAGTAACG
CCAATAGGGACTTTCCATTGACGTCAATGGGTGGAGTATTTACGGTAAACTGCCCACTTGGCA
GTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTA
TTAGTCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATGGGCGGT
AGGCGTGTACGGTGGGAGGTCTATATAAGCAGAGCTGGTTTAGTGAACCGTCAGATCCGCTAG
AGATCCGCGGCCGCATGAAAAGAACCGCTGATGGCAGCGAGTTCGAGAGCCCTAAGAAGAAGC
GGAAGGTGGAGACTCTCCAAGCCTTGGATATGCCAGTGTTGCCTGTAACTGCAACTGAAATCC
GCCAGTATTTGCGGGGGCATGGGATCCCCTTCCAGGATGGTCACAGTTGCCTGCGGGCACTGA
GCCCCTTTGCAGAGTCTTCACAGCTCAAAGGCCAGACTGGTGTTACCACTTCCTTCAGCCTCT
TCATTGACAAGACCACAGGCCACTTTCTCTGCATGACCAGCCTAGCAGAAGGGAGCTGGGAAG
ACTTCCAGGCCAGCGTGGAGGGGCGAGGGGATGGGGCCAGGGAGGGGTTTCTGCTTAGCAAGG
CACCAGAATTTGAGGACAGCGAGGAGGTCCGGAGGATCTGGAACCGAGCAATACCTCTCTGGG
AGCTGCCTGATCAGGAGGAGGTTCAGCTGGCTGATACAATGTTTGGCCTTACCAAGGTTACAG
ATGACACACTCAAGCGTTTCAGTGTGCGATATCTGCGACCTGCTCGCAGTCTTGTCTTCCCTT
GGTTCTCCCCTGGGGGCTCAGGATTACGAGGCCTGAAGCTCCTAGAGGCTAAATGCCAGGGGG
ATGGAGTGAGCTACGAGGAAACCACTATTCCCCGACCCAGCGCCTACCACAATCTGTTTGGAT
TACCACTGATTAGTCGTCGAGATGCTGAGGTGGTACTGACGAGTCGTGAGCTTGACAGCCTGG
CCTTGAACCAGTCCACGGGGCTGCCTACCCTTACTCTACCCCGAGGAACGACCTGCTTACCCC
CTGCCTTACTCCCTTACCTGGAACAGTTCCGGCGGATTGTATTCTGGTTGGGGGATGACCTTC
GGTCCTGGGAAGCCGCCAAGTTGTTTGCACGAAAACTGAACCCCAAACGATGCTTCTTGGTGC
GACCAGGAGACCAGCAACCCCGTCCCCTGGAGGCCCTGAACGGAGGCTTCAATCTTTCTCGTA
TTCTTCGTACCGCCCTGCCTGCCTGGCACAAGTCCATCGTATCTTTCCGGCAGCTTCGGGAGG
AGGTGCTAGGAGAACTGTCAAATGTGGAGCAAGCAGCTGGCCTCCGCTGGAGCCGCTTTCCAG
ACCTCAATCGTATCTTGAAGGGACATCGAAAGGGCGAGCTGACGGTCTTCACAGGGCCAACAG
GCAGTGGAAAGACGACATTCATCAGTGAGTATGCCCTGGATTTGTGTTCCCAGGGGGTGAACA
CACTGTGGGGTAGCTTCGAGATCAGCAATGTGAGACTAGCCCGGGTCATGCTGACACAGTTTG
CCGAGGGGCGGCTGGAAGATCAACTGGACAAATATGATCACTGGGCTGACCGCTTTGAGGACC
TGCCCCTCTATTTCATGACTTTCCATGGACAGCAAAGCATCAGGACTGTAATAGATACAATGC
AACATGCAGTCTACGTCTATGACATTTGTCATGTGATCATCGACAACCTGCAGTTCATGATGG
GTCACGAGCAGCTGTCCACAGACAGGATCGCAGCTCAAGACTACATCATCGGGGTCTTTCGGA
AGTTTGCAACAGACAATAACTGCCATGTGACACTGGTCATTCACCCCCGGAAAGAGGATGATG
ACAAGGAACTGCAGACAGCGTCCATTTTTGGCTCAGCCAAAGCAAGCCAGGAAGCAGACAATG
TTCTGATCCTGCAGGACAGGAAGCTGGTAACCGGGCCAGGGAAACGGTATCTGCAGGTGTCCA
AGAACCGCTTTGATGGAGATGTAGGTGTCTTCCCGCTTGAGTTCAACAAGAACTCCCTCACCT
TCTCCATTCCACCAAAGAACAAGGCCCGGCTCAAGAAGATCAAGGATGACACTGGACCAGTGG
CCAAAAAGCCCTCTTCTGGCAAAAAGGGGGCTACGACACAGAACTCTGAGATTTGCTCAGGCC
AGGCCCCCACTCCCGACCAGCCAGACACCTCCAAGCGTTCAAAGAGCGGCGGCAGCAAAAGAA
CCGCCGACGGCTCAGAGTTCGAGCCCAAAAAGAAGCGGAAAGTGTAAGTTTAAACCCGCTGAT
CAGCCTCGACTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGGGAGGATT
GGGAAGACAATAGCAGGCATGCTGGGGATGCGGTGGGCTCTATGGCTTCTGAGGCGGAAAGAA
CCAGCTGGGGCTCGATACCGTCGACCTCTAGCTAGAGCTTGGCGTAATCATGGTCATAGCTGT
TTCCTGTGTGAAATTGTTATCCGCTCACAATTCCACACAACATACGAGCCGGAAGCATAAAGT
GTAAAGCCTAGGGTGCCTAATGAGTGAGCTAACTCACATTAATTGCGTTGCGCTCACTGCCCG
CTTTCCAGTCGGGAAACCTGTCGTGCCAGCTGCATTAATGAATCGGCCAACGCGCGGGGAGAG
GCGGTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGACGCTCAA
GTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGTTTTTTTGTTTGCA
AGCAGCAGATTACGCGCAGAAAAAAAGGATCTCAA
pCMV-NLS-Trex2-NLS Plasmid Sequence:
>SEQ ID NO: 14
GAAGATCCTTTGATCTTTTCTACGGGGTCTGACACTCAGTGGAACGAAAACTCACGTTAAGGG
ATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGTGTCA
CGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCG
CGCACATTTCCCCGAAAAGTGCCACCTGACGTCGACGGATCGGGAGATCGATCTCCCGATCCC
CTAGGGTCGACTCTCAGTACAATCTGCTCTGATGCCGCATAGTTAAGCCAGTATCTGCTCCCT
GCTTGTGTGTTGGAGGTCGCTGAGTAGTGCGCGAGCAAAATTTAAGCTACAACAAGGCAAGGC
TTGACCGACAATTGCATGAAGAATCTGCTTAGGGTTAGGCGTTTTGCGCTGCTTCGCGATGTA
CGGGCCAGATATACGCGTTGACATTGATTATTGACTAGTTATTAATAGTAATCAATTACGGGG
TCATTAGTTCATAGCCCATATATTGAGTTCCGCGTTACATAACTTACGGTAAATGGCCCGCCT
GGCTGACCGCCCAACGACCCCCGCCCATTGACGTCAATAATGACGTATGTTCCCATAGTAACG
CCAATAGGGACTTTCCATTGACGTCAATGGGTGGAGTATTTACGGTAAACTGCCCACTTGGCA
GTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTA
TTAGTCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATGGGCGGT
AGGCGTGTACGGTGGGAGGTCTATATAAGCAGAGCTGGTTTAGTGAACCGTCAGATCCGCTAG
AGATCCGCGGCCGCATGAAAAGAACCGCTGATGGCAGCGAGTTCGAGAGCCCTAAGAAGAAGC
GGAAGGTGATGTCCGAACCACCTAGAGCCGAGACCTTCGTGTTCCTGGACCTGGAGGCCACAG
GCCTGCCCAACATGGACCCCGAGATCGCCGAAATCAGCCTGTTTGCCGTGCACAGAAGCAGCC
TGGAAAACCCTGAAAGAGATGACAGCGGCAGCCTGGTGCTGCCCAGAGTGCTGGACAAGCTGA
CACTGTGTATGTGCCCCGAACGGCCTTTCACCGCCAAGGCCTCTGAAATCACCGGACTGTCTT
CTGAGAGCCTGATGCACTGCGGCAAGGCCGGCTTTAACGGCGCTGTGGTGCGGACCCTGCAGG
GCTTTCTGTCCAGACAGGAGGGCCCCATCTGCCTGGTCGCCCACAACGGCTTCGATTACGACT
TCCCCCTGCTCTGCACCGAGCTGCAAAGACTGGGCGCCCACCTGCCACAGGACACCGTGTGTC
TGGATACACTGCCCGCCCTGCGGGGCCTGGACAGAGCCCATAGCCACGGCACCAGGGCCCAGG
GCAGAAAGAGCTACAGCCTTGCTTCTCTGTTCCACCGGTACTTCCAGGCCGAGCCCTCTGCCG
CCCACAGCGCCGAGGGAGATGTGCACACACTGCTGCTGATCTTCCTGCACAGAGCTCCTGAAC
TGCTGGCCTGGGCTGATGAGCAGGCCCGCAGCTGGGCTCACATCGAGCCTATGTACGTGCCTC
CTGACGGCCCTTCCCTGGAAGCCAGCGGCGGCAGCAAAAGAACCGCCGACGGCTCAGAGTTCG
AGCCCAAAAAGAAGCGGAAAGTGTAAGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACAATAGCAGGCATG
CTGGGGATGCGGTGGGCTCTATGGCTTCTGAGGCGGAAAGAACCAGCTGGGGCTCGATACCGT
CGACCTCTAGCTAGAGCTTGGCGTAATCATGGTCATAGCTGTTTCCTGTGTGAAATTGTTATC
CGCTCACAATTCCACACAACATACGAGCCGGAAGCATAAAGTGTAAAGCCTAGGGTGCCTAAT
GAGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGCGCAGAA
AAAAAGGATCTCAA
pCMV-NLS-FEN1-NLS Plasmid Sequence:
>SEQ ID NO: 15
GAAGATCCTTTGATCTTTTCTACGGGGTCTGACACTCAGTGGAACGAAAACTCACGTTAAGGG
ATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGTGTCA
CGCTCGTCGTTTGGTATGGCTTCATTCAGCTCCGGTTCCCAACGATCAAGGCGAGTTACATGA
TCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCA
TCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCG
CGCACATTTCCCCGAAAAGTGCCACCTGACGTCGACGGATCGGGAGATCGATCTCCCGATCCC
CTAGGGTCGACTCTCAGTACAATCTGCTCTGATGCCGCATAGTTAAGCCAGTATCTGCTCCCT
GCTTGTGTGTTGGAGGTCGCTGAGTAGTGCGCGAGCAAAATTTAAGCTACAACAAGGCAAGGC
TTGACCGACAATTGCATGAAGAATCTGCTTAGGGTTAGGCGTTTTGCGCTGCTTCGCGATGTA
CGGGCCAGATATACGCGTTGACATTGATTATTGACTAGTTATTAATAGTAATCAATTACGGGG
TCATTAGTTCATAGCCCATATATTGAGTTCCGCGTTACATAACTTACGGTAAATGGCCCGCCT
GGCTGACCGCCCAACGACCCCCGCCCATTGACGTCAATAATGACGTATGTTCCCATAGTAACG
CCAATAGGGACTTTCCATTGACGTCAATGGGTGGAGTATTTACGGTAAACTGCCCACTTGGCA
GTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTA
TTAGTCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATGGGCGGT
AGGCGTGTACGGTGGGAGGTCTATATAAGCAGAGCTGGTTTAGTGAACCGTCAGATCCGCTAG
AGATCCGCGGCCGCATGAAAAGAACCGCTGATGGCAGCGAGTTCGAGAGCCCTAAGAAGAAGC
GGAAGGTGATGGGAATTCAAGGCCTGGCCAAACTAATTGCTGATGTGGCCCCCAGTGCCATCC
GGGAGAATGACATCAAGAGCTACTTTGGCCGTAAGGTGGCCATTGATGCCTCTATGAGCATTT
ATCAGTTCCTGATTGCTGTTCGCCAGGGTGGGGATGTGCTGCAGAATGAGGAGGGTGAGACCA
CCAGCCACCTGATGGGCATGTTCTACCGCACCATTCGCATGATGGAGAACGGCATCAAGCCCG
TGTATGTCTTTGATGGCAAGCCGCCACAGCTCAAGTCAGGCGAGCTGGCCAAACGCAGTGAGC
GGCGGGCTGAGGCAGAGAAGCAGCTGCAGCAGGCTCAGGCTGCTGGGGCCGAGCAGGAGGTGG
AAAAATTCACTAAGCGGCTGGTGAAGGTCACTAAGCAGCACAATGATGAGTGCAAACATCTGC
TGAGCCTCATGGGCATCCCTTATCTTGATGCACCCAGTGAGGCAGAGGCCAGCTGTGCTGCCC
TGGTGAAGGCTGGCAAAGTCTATGCTGCGGCTACCGAGGACATGGACTGCCTCACCTTCGGCA
GCCCTGTGCTAATGCGACACCTGACTGCCAGTGAAGCCAAAAAGCTGCCAATCCAGGAATTCC
ACCTGAGCCGGATTCTGCAGGAGCTGGGCCTGAACCAGGAACAGTTTGTGGATCTGTGCATCC
TGCTAGGCAGTGACTACTGTGAGAGTATCCGGGGTATTGGGCCCAAGCGGGCTGTGGACCTCA
TCCAGAAGCACAAGAGCATCGAGGAGATCGTGCGGCGACTTGACCCCAACAAGTACCCTGTGC
CAGAAAATTGGCTCCACAAGGAGGCTCACCAGCTCTTCTTGGAACCTGAGGTGCTGGACCCAG
AGTCTGTGGAGCTGAAGTGGAGCGAGCCAAATGAAGAAGAGCTGATCAAGTTCATGTGTGGTG
AAAAGCAGTTCTCTGAGGAGCGAATCCGCAGTGGGGTCAAGAGGCTGAGTAAGAGCCGCCAAG
GCAGCACCCAGGGCCGCCTGGATGATTTCTTCAAGGTGACCGGCTCACTCTCTTCAGCTAAGC
GCAAGGAGCCAGAACCCAAGGGATCCACTAAGAAGAAGGCAAAGACTGGGGCAGCAGGGAAGT
TTAAAAGGGGAAAAAGCGGCGGCAGCAAAAGAACCGCCGACGGCTCAGAGTTCGAGCCCAAAA
AGAAGCGGAAAGTGTAAGTTTAAACCCGCTGATCAGCCTCGACTGTGCCTTCTAGTTGCCAGC
CATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGCTGGGGATG
CGGTGGGCTCTATGGCTTCTGAGGCGGAAAGAACCAGCTGGGGCTCGATACCGTCGACCTCTA
GCTAGAGCTTGGCGTAATCATGGTCATAGCTGTTTCCTGTGTGAAATTGTTATCCGCTCACAA
TTCCACACAACATACGAGCCGGAAGCATAAAGTGTAAAGCCTAGGGTGCCTAATGAGTGAGCT
AACTCACATTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGAT
CTCAA.

What is claimed is:

1. A method for performing strand displacement-based gene editing in a DNA sequence, the method comprising:
bringing a double-stranded DNA sequence into contact with a sequence-specific nucleic acid nickase to create a nick on the double-stranded DNA sequence;
digesting from the nick with an exonuclease to generate a free single-stranded DNA;
bringing the free single-stranded DNA into contact with an invader DNA and a human mitochondrial DNA helicase Twinkle enzyme; wherein the invader DNA is a single-stranded or double-stranded DNA, and at least one strand of the invader DNA comprises a glue point complementary to the free single-stranded DNA and a displacement template containing a desired nucleotide alteration;
thereby subjecting the glue point of the invader DNA to complementary pairing with the free single-stranded DNA;
thereby subjecting the displacement template of the invader DNA to a strand displacement reaction with an endogenous DNA strand adjacent to a nick site; and
performing DNA repair/replication to introduce the desired nucleotide alteration into the double-stranded DNA sequence.

2. The method according to claim 1, wherein a process of the DNA repair comprises: (1) performing the DNA repair using the displacement template as a template to form a product with the desired nucleotide alteration on two DNA strands; and (2) repairing the nick on DNA.

3. The method according to claim 1, wherein the process of the DNA repair further comprises excising the endogenous DNA strand adjacent to the nick site using a structure-specific nuclease after the strand displacement reaction; wherein the structure-specific nuclease is preferably FEN1.

4. The method according to claim 1, wherein the sequence-specific nucleic acid nickase is a CRISPR-associated protein (Cas) polypeptide having a nickase activity, a zinc finger nuclease (ZFN), a transcription activator-like effector nuclease (TALEN), a meganuclease, a sequence-specific endonuclease, or a variant, fragment and combination thereof; or
wherein the sequence-specific nucleic acid nickase is a CRISPR-associated protein (Cas) polypeptide having a nickase activity, the Cas polypeptide is selected from Cas3, Cas4, Cas5, Cas5e, Cas6, Cas6e, Cas6f, Cas7, Cas8a1, Cas8a2, Cas8b, Cas8c, Cas9, Cas10, Cas10d, Cas12, Cas13, Cas14, CasX, CasF, CasG, CasH, Csy1, Csy2, Csy3, Cse1, Cse2, Cse3, Cse4, Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, TranC, and a variant, fragment or any combination thereof, or
wherein the sequence-specific nucleic acid nickase comprises a mutant comprising a D10A mutation or an H840A mutation corresponding to the amino acid sequence set forth in SEQ ID NO: 1.

5. The method according to claim 1, wherein the Twinkle enzyme is a naturally occurring or truncated Twinkle enzyme; and the Twinkle enzyme comprises a carboxyl-terminal domain (CTD) having an amino acid sequence set forth in SEQ ID NO: 2; and/or
wherein a mitochondrial localization signal is removed from the Twinkle enzyme, and then a nuclear localization signal (NLS) is fused to an amino terminus and/or carboxyl terminus of the Twinkle enzyme.

6. The method according to claim 1, wherein the exonuclease is a 3′ exonuclease or a 5′ exonuclease or a combination thereof; wherein the 3′ exonuclease is preferably Trex2; and the 5′ exonuclease is preferably mExol or T5 exonuclease.

7. The method according to claim 1, wherein the desired nucleotide alteration comprises a substitution, insertion or deletion of one or more nucleotides.

8. A strand displacement-based gene editing system, comprising components of the strand displacement-based gene editing system and/or an expression construct comprising a nucleotide sequence that encodes the components of the strand displacement-based gene editing system, the components of the strand displacement-based gene editing system comprising:
i) a sequence-specific nucleic acid nickase or a domain thereof that is guided by a guide RNA (sgRNA) sequence to a target DNA;
ii) an exonuclease or a domain thereof;
iii) a human mitochondrial DNA helicase Twinkle enzyme or a domain thereof; and
iv) a single-stranded or double-stranded invader DNA, wherein at least one strand of the invader DNA comprises a glue point complementary to a free single-stranded DNA generated under action of a nickase and the exonuclease, and a displacement template containing a desired nucleotide alteration;
the components being independent of each other, or at least two of the components are linked via a linker to form a fusion protein.

9. The strand displacement-based gene editing system according to claim 8, wherein the components of the strand displacement-based gene editing system further comprise:
v) a structure-specific nuclease;
wherein the structure-specific nuclease is preferably FEN1.

10. The strand displacement-based gene editing system according to claim 8, wherein the sequence-specific nucleic acid nickase is a CRISPR-associated protein (Cas) polypeptide having a nickase activity, a zinc finger nuclease (ZFN), a transcription activator-like effector nuclease (TALEN), a meganuclease, a sequence-specific endonuclease, or a variant, fragment and combination thereof; or
wherein the sequence-specific nucleic acid nickase is the CRISPR-associated protein (Cas) polypeptide having a nickase activity; and the components of the strand displacement-based gene editing system further comprise: vi) a guide RNA that guides a corresponding Cas polypeptide to a target nucleotide region; or
wherein the sequence-specific nucleic acid nickase is the CRISPR-associated protein (Cas) polypeptide having a nickase activity; and the components of the strand displacement-based gene editing system further comprise: vi) a guide RNA that guides a corresponding Cas polypeptide to a target nucleotide region, the Cas polypeptide is selected from Cas3, Cas4, Cas5, Cas5e, Cas6, Cas6e, Cas6f, Cas7, Cas8a1, Cas8a2, Cas8b, Cas8c, Cas9, Cas10, Cas10d, Cas12, Cas13, Cas14, CasX, CasF, CasG, CasH, Csy1, Csy2, Csy3, Cse1, Cse2, Cse3, Cse4, Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, Cu1966, TranC, and a variant, fragment or any combination thereof, or
wherein the sequence-specific nucleic acid nickase comprises a mutant comprising a D10A mutation or an H840A mutation corresponding to the amino acid sequence set forth in SEQ ID NO: 1.

11. The strand displacement-based gene editing system according to claim 8, wherein the Twinkle enzyme is a naturally occurring or truncated Twinkle enzyme; and the Twinkle enzyme comprises a carboxyl-terminal domain (CTD) having an amino acid sequence set forth in SEQ ID NO: 2.

12. The strand displacement-based gene editing system according to claim 8, wherein the exonuclease is a 3′ exonuclease or a 5′ exonuclease or a combination thereof; wherein the 3′ exonuclease is preferably Trex2; and the 5′ exonuclease is preferably mExol or T5 exonuclease.

13. The strand displacement-based gene editing system according to claim 8, wherein a nuclear localization signal (NLS) is fused to an amino or carboxyl terminus of at least one of the components; and/or
wherein the linker comprises an amino acid sequence (GGGS)n, (GGGGS)n, (G)n, (EAAAK)n, (GGS)n, (SGGS)n or SGSETPGTSESATPES, or a (XP)n motif or a combination thereof, wherein n is independently an integer of 1 to 30, and wherein X is any amino acid.

14. The strand displacement-based gene editing system according to claim 8, wherein each of the components of the strand displacement-based gene editing system is recruited via a recruitment system that is a MCP-MS2 system or a GCN4-ScFv system.

15. A host cell, wherein the host cell comprises the strand displacement-based gene editing system according to claim 8.

16. A method for generating at least one cell that is genetically modified, wherein the method comprises introducing the strand displacement-based gene editing system according to claim 8 into the at least one cell to cause a substitution, insertion or deletion of one or more nucleotides within a target nucleotide editing region in the at least one cell.

17. The method according to claim 16, wherein the method further comprises a step of screening the at least one cell for a cell having one or more desired nucleotide substitutions.

18. The method according to claim 16, wherein the cell is derived from a prokaryote such as bacteria; or an eukaryote such as plants, fungi or vertebrates; or
wherein the cell is derived from vertebrates, the vertebrates are mammals such as human, mouse, rat, monkey, dog, pig, sheep, cattle, and cat; or
wherein the cell is derived from plants, the plants are crop plants, such as wheat, rice, corn, soybean, sunflower, sorghum, oilseed rape, alfalfa, cotton, barley, millet, sugar cane, tomato, tobacco, cassava or potato.

19. A method for gene or genome editing, detection and/or diagnosis of a target nucleotide, editing of a target nucleotide sequence to modify an organism or a non-human organism, or treatment of a disease, wherein the method comprises utilizing the strand displacement-based gene editing system according to claim 8.

20. A kit, wherein the kit comprises the strand displacement-based gene editing system according to claim 8.
